(19) United States
(12) Patent Application Publication
(10) Pub. No.: US 2024/0309342 A1
(43) Pub. Date: Sep. 19, 2024

(54) QUORUM QUENCHING ENZYME, AND METHODS OF USING SAME

(71) Applicant: MIGAL GALILEE RESEARCH INSTITUTE LTD., Kiryat Shmona (IL)

(72) Inventors: Livnat AFRIAT-JURNOU, Kibbutz Kfar Szold (IL); Itai SHARON, Kibbutz Mayan Barukh (IL)

(73) Assignee: MIGAL GALILEE RESEARCH INSTITUTE LTD., Kiryat Shmona (IL)

(21) Appl. No.: 18/290,897

(22) PCT Filed: Jul. 21, 2022
(86) PCT No.: PCT/IL2022/050792

§ 371 Date: Jan. 22, 2024

Related U.S. Application Data
(60) Provisional application No. 63/224,462, filed on Jul. 22, 2021.

(51) Int. Cl. C12N 9/18; A23C 9/12

(52) U.S. Cl. CPC C12N 9/18; A23C 9/1216; C12Y 301/01081

(57) ABSTRACT

The present invention is directed to an isolated polypeptide being a quorum-quenching N-acyl-homoserine lactonase, compositions including same, and methods of using same, such as for reducing or inhibiting formation of load of an organic-based contaminant.

### CROSS-REFERENCE TO RELATED APPLICATION

The present invention claims the benefit of priority of U.S. Provisional Patent Application No. 63/224,462, titled “QUORUM QUENCHING ENZYME, AND METHODS OF USING SAME”, filed 22 Jul. 2021, the contents of which are incorporated herein by reference in their entirety.

### FIELD OF THE INVENTION

The present invention is in the field of biochemistry and microbiology.

### BACKGROUND

In light of the spread of bacterial resistance to existing antibiotic agent, there is a constant and urgent need for novel antibacterial agents, operating through alternative bacterial killing or inhibition mechanisms. Promising strategies have recently focused on interfering bacterial communication pathways, also known as “quorum sensing” (QS), a form of cell-to-cell communication that enable bacteria to synchronize the regulation of gene expression in correlation with population density. Specifically, QS bacteria release chemical molecules termed “autoinducers” which in many instances affect gene expression in neighboring bacterial cells. Both Gram-positive and Gram-negative bacteria use quorum sensing communication circuits to regulate varied physiological activities. Processes modulated through quorum sensing include virulence, competence, conjugation, antibiotic production, motility, and biofilm formation. In general, Gram-negative bacteria use acylated homoserine lactones (AHLs) as autoinducers, and Gram-positive bacteria use processed oligo-peptides (DPD). Additionally, data suggesting that bacterial autoinducers also activate specific biological responses of the host organism is mounting.

The marine environment presents a habitat with vast potential as a resource for microorganisms and novel enzymes with unique biochemical properties and novel activities. Quorum quenching enzymes hydrolyze bacterial QS signaling molecules, such as AHLs.

Identification and use of substances and/or enzymes that interfere and/or disrupt in quorum sensing pathways of pathogenic bacteria and thus block their virulence, is of high importance.

### SUMMARY

According to a first aspect, there is provided an isolated polypeptide comprising an amino acid sequence as set forth in SEQ ID NO: 3, or a functional analog having at least 95% homology or identity thereto.

According to another aspect, there is provided a polynucleotide encoding the isolated polypeptide of the invention.

According to another aspect, there is provided an artificial nucleic acid molecule or vector comprising the polynucleotide disclosed herein.

According to another aspect, there is provided a cell comprising any one of: (a) the isolated polypeptide of the invention; (b) the herein disclosed polynucleotide; (c) the herein disclosed artificial nucleic acid molecule or vector; and (d) any combination of (a) to (c).

According to another aspect, there is provided an extract obtained or derived from the herein disclosed cell.

According to another aspect, there is provided a composition comprising any one of: (a) the isolated polypeptide of the invention; (b) the herein disclosed polynucleotide; (c) the herein disclosed artificial nucleic acid molecule or vector; (d) the herein disclosed; (e) the herein disclosed extract; and (f) any combination of (a) to (d), and an acceptable carrier.

According to another aspect, there is provided an article comprising the isolated polypeptide of the invention.

According to another aspect, there is provided a method of inhibiting or reducing a formation of load of organic-based contaminant on or within an article, comprising coating or incorporating a composition comprising the isolated polypeptide of the invention to the article, thereby inhibiting or reducing a formation of load of organic-based contaminant on or within the article.

According to another aspect, there is provided a method of inhibiting or reducing a formation of load of organic-based contaminant in a composition, the method comprising contacting the composition with an effective amount of the isolated polypeptide of the invention.

In some embodiments, the isolated polypeptide comprises an amino acid sequence as set forth in SEQ ID NO: 1, or a functional analog having at least 95% homology or identity thereto.

In some embodiments, the isolated polypeptide is characterized by having an activity at pH ranging from 7.5 to 9.5.

In some embodiments, the isolated polypeptide is characterized by having an activity at a salinity ranging from 0 to 5 M.

In some embodiments, the isolated polypeptide is characterized by having an activity at a temperature ranging from 1 to 60° C.

In some embodiments, the activity comprises at least 50% activity compared to an optimal activity control.

In some embodiments, the isolated polypeptide is characterized by being stable at temperature ranging from 1 to 85° C.

In some embodiments, the isolated polypeptide is characterized by being stable at a pH ranging from 4.5 to 11.0.

In some embodiments, the isolated polypeptide is characterized by being stable at a salinity ranging from 0 to 5 M.

In some embodiments, stable is comprising or maintaining at least 50% of the activity compared to optimal conditions.

In some embodiments, the activity comprises ester bond hydrolysis.

In some embodiments, the ester bond is an ester bond of a homoserine lactone ring.

In some embodiments, the homoserine lactone is an acylated homoserine lactone.

In some embodiments, the isolated polypeptide is a quorum-quenching N-acyl-homoserine lactonase.

In some embodiments, the polynucleotide comprises a nucleic acid sequence being codon optimized for expression in a cell.

In some embodiments, the nucleic acid sequence being codon optimized for expression in cell comprises a sequence set forth in SEQ ID NO: 2.

In some embodiments, the cell is a bacterial cell or a fungal cell.

In some embodiments, the artificial nucleic acid molecule or vector is a plasmid or an expression vector.

In some embodiments, the cell is any one of: a unicellular organism, a cell of a multicellular organism, and a cell in a culture.

In some embodiments, the cell is a transgenic cell, a transformed cell, or a transfected cell.

In some embodiments, the extract comprises any one of: (a) the isolated polypeptide of the invention; (b) the herein disclosed polynucleotide; (c) the herein disclosed artificial nucleic acid molecule or vector; and (d) any combination of (a) to (c).

In some embodiments, the isolated polypeptide is bound to a surface of the article.

In some embodiments, bound is covalently bound.

In some embodiments, the article is configured to storing or transferring a liquid.

In some embodiments, incorporating comprises binding the isolated polypeptide to a surface of the article.

In some embodiments, binding comprises covalently binding.

In some embodiments, the liquid comprises water.

In some embodiments, the article comprises any one of: a tubing, a capsule, and a container.

In some embodiments, the composition is an edible composition.

In some embodiments, the edible composition is a dairy product.

In some embodiments, the organic-based contaminant comprises biofilm.

In some embodiments, the biofilm is produced by Pseudomonas fluorescens.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes an illustration of evolutionary relationships of previously characterized phosphotriesterase like-lactonase (PLLs) and the newly identified homologs from metagenomics libraries. Phosphotriesterase (PTE) homologs were used as an outgroup. The evolutionary history was inferred using the Neighbor-Joining method. The optimal tree with the sum of branch length=5.56804021 is shown. The percentage of replicate trees in which the associated taxa clustered together in the bootstrap test (100 replicates) are shown next to the branches. The tree is drawn to scale, with branch lengths in the same units as those of the evolutionary distances used to infer the phylogenetic tree. The evolutionary distances were computed using the Poisson correction method and are in the units of the number of amino acid substitutions per site. The analysis involved 26 amino acid sequences. All positions containing gaps and missing data were eliminated. There were a total of 305 positions in the final dataset. Evolutionary analyses were conducted in MEGA7.

FIGS. 2A-2C include multiple sequence alignment of previously characterized PTE-Like-Lactonase (PLLs) and the newly identified homologs from marine metagenomes, and structural overlay of a computational model of marine originated lactonase related protein (moLRP) and a thermostable quorum-quenching lactonase from Geobacillus kaustophilus GKL (pdb 3ojg). (2A) The new marine PLL aligned well with the PLL family using the MUSCLE algorithm. The previously characterized PLLs are: SsoPox from Sulfolbus solfataricus (WP 009988477.1), SacPox from Sulfolbus acidocaldarius (WP 011278935.1), SisLac from Sulfolbus islandicus (WP 012710474.1), PPH from M. tuberculosis (ACF57854.1) and AhlA from Rhodococcus erythropolis (ACF57853.1), GKL from Geobacillus kaustophilus and PTE from Pseudomonas diminuta (IHZY; Benning et al., 2001, Biochemistry). (2B) The backbones of the homology modeling of moLRP (arrowhead) and solved structure of GKL (asterisk) with N-butyryl-DL-homoserine lactone (pdb 3ojg; Chow et al., 2010, JBC, sharing 40% identity). The active site metal atoms (arrows) and N-butyryl-DL-homoserine lactone in the active site of lactonase from GKL. (2C) Overlay of the binuclear catalytic center. Five of the zinc-ligating residues (His23, -25, -178, and -266, and Asp301) align perfectly.

FIG. 3 includes a graph showing Michaelis-Menten analyses of the lactonase activity of the newly identified moLRP with C4-HSL, at E₀=0.4 μM, 27° C.

FIGS. 4A-4F include radar charts showing that the biochemical properties of moLRP indicate it is active and stable over a wide range of temperatures, pH, and salinity levels. (4A-4C) Presenting radar charts of the relative activity of moLRP and PPH with TBBL (thio-buthyl-γ-butyric-lactone; a chromogenic lactone substrate) at different temperatures (4A), pH (4B), and salinity levels (4C). (4D-4F) Presenting radar charts of the residual activity of moLRP and PPH after 1 h incubation of the enzymes in different conditions of temperatures (4D), pH (4E), and salinity (4F), and then testing activity at optimal conditions. Enzymes concentrations are as follow: in (4B-4C), and (4F) 0.3 μM of moLRP were used. In (4A, 4D) and (4E): 0.025, 0.15, 2.5 UM of moLRP were used respectively. In (4A) and (4D) 1 μM of PPH was used, in (4C) and (4F): 2 μM of PPH were used. In (4B) and (4E) 1.75 μM and 1.4 μM of PPH were used, respectively.

FIGS. 5A-5C include vertical bar graphs showing that moLRP maintains its activity for at least 60 hours in culture and presents higher inhibition of the extracellular proteolytic activity in skim milk cultures of P. fluorescents, compared to PPH. Bacterial cultures of P. fluorescents were grown in skim milk and incubated without or with 1 μM purified enzyme (moLRP or PPH). Skim milk medium without the bacteria was used as a negative control (i.e., control). (5A) Purified enzymes (1 μM) PPH or moLRP were added to P. fluorescens cultures and incubated at 28° C., under 170 rpm. moLRP activity was tested (with 0.05 mM TBBL) in supernatants of each culture, every hour. Residual activity was calculated comparing the activity of each supernatants to that of the corresponding enzyme in its activity buffer. (5B) Extracellular proteinase activity of P. fluorescens in the presence of purified enzymes. Proteinase activity was quantified using azocasein assay at OD440. Data are presented as means±SD (n=3). Statistical significance according to one way ANOVA (****p<0.0001). P. fluorescens alone use as control. (5C) Biofilm formation of P. fluorescens in the presence of purified enzymes. Biofilm formation quantified by crystal violet assay at OD590. Data are presented as means±SD (control, n=5; PPH/moLRP, n=3). Statistical significance according to one way ANOVA (ns, non-significant; ****p<0.0001). P. fluorescens infection alone uses as control.

FIGS. 6A-6C include a micrograph and graphs showing that moLRP inhibits milk sedimentation in P. fluorescens treated milk-based cultures. (6A) Skim-milk cultures of P. fluorescens, at 28° C., incubated with or without purified enzymes: Skim milk without bacteria as negative control (a); P. fluorescens alone (b); P. fluorescens with 1 μM wtPPH (c); and P. fluorescens with 1 μM moLRP (d). Pictures were taken after 4 days of incubation. (6B-6C) Particle velocity distributions was analyzed by LUMiFuge of P. fluorescens treated milk-based cultures. (6B) Integrated transmission percentage in milk based bacterial cultures with and without purified moLRP over time. (Data are presented as means±SD (n=3). Statistical significance according to one way ANOVA (****p<0.0001). (6C) Mean values of light transmission were examined in test tubes of bacterial cultures after 6 hours. Milk medium alone was used as a negative control.

FIG. 7 includes a multiple sequence alignment of previously characterized PLLs and putative PLLs from marine metagenomes. The new putative marine PLL aligned with the PLL family using the MUSCLE algorithm. The previously characterized PLLs are: SsoPox from Sulfolbus solfataricus (WP 009988477.1), SacPox from Sulfolbus acidocaldarius (WP 011278935.1), SisLac from Sulfolbus islandicus (WP 012710474.1), PPH from M. tuberculosis (ACF57854.1) and AhlA from Rhodococcus erythropolis (ACF57853.1), GKL from Geobacillus kaustophilus, and PTE from Pseudomonas Diminuta (1HZY).

FIGS. 8A-8F include graphs showing detailed comparison of the biochemical properties of moLRP to PPH, presented above in FIGS. 4E-4F. (8A) The optimal temperature for the AHL-degrading activity of purified moLRP and PPH. The enzymes activities were determined by measuring the rate of enzymatic hydrolysis of TBBL in the temperature range from 5 to 90° C. Error bars indicate standard deviation. 100% relative activity was defined as the activity at 45° C. for moLRP and 35° C. for PPH. (8B) For thermal stability, the enzymes in reaction buffers were incubated at temperatures ranging from 5 to 90° C. for one hour. After incubation, the activity was measured. 100% relative activity was defined for moLRP's activity at 15° C. and PPH at 85° C., and error bars indicate standard deviation. (8C) The optimal pH was determined by enzymes activities in a different pH (3.5 to 11). 100% relative activity was defined as the activity at pH 8 for moLRP and pH 9 for PPH. Error bars indicate standard deviation. (8D) pH stability was tested after pre-incubation of the enzymes for one hour without a substrate at 4° C., in buffers with a range of pH values (3.5-11), and error bars indicate standard deviation. Residual activity was measured at an optimal pH and at a constant temperature of 25° C. (8E) The optimal NaCl concentration was determined by enzymes activities in a wide range of NaCl concentrations (0 to 5 M). Error bars indicate standard deviation. (8F) Enzyme stability in different salinity levels was tested after pre-incubation of the enzymes for one hour without a substrate at 4° C. in buffers with a range of NaCl concentrations (0 to 5 M). Residual activity was measured at an optimal pH and a constant temperature of 25° C. relative to 5 M NaCl for moLRP and 2 M for PPH, and error bars indicate standard deviation.

FIGS. 9A-9B include a growth curve and a vertical bar graph. (9A) Relative expression levels of aprX gene in cultures in the presence of purified moLRP and its activity buffer, as control. The transcription of QS-regulated gene aprX (proteinase gene) was normalized to the housekeeping gene 16S rRNA. Data points represent the means of three biological replicates±standard error. Statistical significance according to one-way ANOVA comparison (Tukey's multiple comparisons test) (*p value<0.05). (9B) Growth curve measured for 69 hours. Cultures of P. fluorescens alone, with enzyme activity buffer or with purified (1 μM) moLRP, grown in 200 μL LB media in 96 well plate to enable absorbance detection at OD₆₀₀.

### DETAILED DESCRIPTION

The present invention, in some embodiments, is directed to an isolated polypeptide having lactonase activity and being derived from a marine origin, a polynucleotide encoding same, including methods of using same.

### Polypeptide

According to some embodiments, there is provided an isolated polypeptide comprising the amino acid sequence: MASINSVLGPLDTADIGYTLSHEHVMVTSAGIQYVYPEFIDREGSISKGIADLKTA YGEGLRTIVDVTTIDLGRDIRMLEQVSRESGINIICATGVWRDIPRVFWSASPDMV APLFIREIEEGIEGTGIKAGIIKVANDMGGVTAEGEIILRAAARAQKATGVPISTHT WAPERVGEQQVRIFEDEGVDLNRVYVGHSNDTTDTDYLIGLLEKGVWIGLDRYP GRQTEHTPDWIGRTETAKKLIDAGYGHRIMLGHDWSVTLSIASKEMQEQRLKYN PDGYLFITRNVVPKLLELGATDEDIQNIFVNNPRNFFEGS (SEQ ID NO: 1), or a functional analog having at least 95%, 96%, 97%, 98%, 99%, or 100% homology or identity thereto, or any value and range therebetween. Each possibility represents a separate embodiment of the invention.

As used herein, the terms “peptide”, “polypeptide” and “protein” refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, a peptide comprises a chain of 2 to 50 amino acids.

In some embodiments, a peptide is up to 50 amino acids long.

In some embodiments, the terms “polypeptide” and “protein” are used interchangeably.

In some embodiments, a “polypeptide” and/or a “protein” comprises at least 50 amino acids.

In some embodiments, a “polypeptide” and/or a “protein” comprises multiple peptide subunits.

In some embodiments, the isolated polypeptide is characterized by having or comprises an activity at pH of at least 7.4, 7.5, 7.6, 7.7, 7.8, 7.9, 8.0, 8.2, 8.5, 8.6, 8.8, 9.0, 9.1, 9.3, or 9.5, or any value and range therebetween. Each possibility represents a separate embodiment of the invention.

In some embodiments, the isolated polypeptide is characterized by having or comprises an activity at pH ranging from 2 to 9, 2.5 to 8.5, 2.8 to 9.7, 3 to 8.5, 3.5 to 9, 4 to 8, 4.5 to 9.5, 5 to 9, 5.5 to 9.5, 6 to 8.5, 6 to 9.5, 7.5 to 9.5, 7.4 to 9.6, 7.5 to 9.3, 7.6 to 9.3, 8.0 to 9.6, or 7.8 to 9.4. Each possibility represents a separate embodiment of the invention.

In some embodiments, the isolated polypeptide is characterized by having or comprises an activity at a salinity of at least 0.1 M, 0.3 M, 0.5 M, 0.7 M, 0.9 M, 1.0 M, 1.3 M, 1.5 M, 2.0 M, 2.5 M, 3.0 M, 3.5 M, 4.0 M, 4.5 M, 5.0 M, or any value and range therebetween. Each possibility represents a separate embodiment of the invention.

In some embodiments, the isolated polypeptide is characterized by having or comprises an activity at a salinity ranging from 0 to 5 M, 0.1 to 4.9 M, 0.5 to 5.5 M, 0.1 to 4 M, 0.3 to 4.8 M, 0.7 to 3.8 M, or 0.5 to 3 M. Each possibility represents a separate embodiment of the invention.

In some embodiments, the isolated polypeptide is characterized by having or comprises an activity at a temperature of at least 0.1° C., 1° C., 5° C., 10° C., 15° C., 20° C., 25° C., 30° C., 35° C., 40° C., 45° C., 50° C., 55° C., 60° C., 65° C., or any value and range therebetween. Each possibility represents a separate embodiment of the invention.

In some embodiments, the isolated polypeptide is characterized by having or comprises an activity at a temperature ranging from 1 to 60° C., 0.1 to 50° C., 0.5 to 40° C., 0.8 to 55° C., 2 to 60° C., 7 to 55° C., 5 to 56° C., 20 to 60° C., 22 to 49° C., 25 to 65° C., or 10 to 70° C. Each possibility represents a separate embodiment of the invention.

As used herein, the term “activity” encompasses or refers to ester bond hydrolysis. In some embodiments, an ester comprises a cyclic ester. In some embodiments, an ester comprises a lactone. In some embodiments, the isolated polypeptide of the invention is characterized by being capable of hydrolyzing an ester bond. In some embodiments, the isolated polypeptide of the invention is characterized by being capable of hydrolyzing a cyclic ester bond. In some embodiments, the isolated polypeptide of the invention is characterized by being an ester bond hydrolyzing enzyme. In some embodiments, the isolated polypeptide of the invention is characterized by being a cyclic bond hydrolyzing enzyme. In some embodiments, the isolated polypeptide of the invention is an esterase. In some embodiments, the isolated polypeptide of the invention is a lactonase. In some embodiments, the isolated polypeptide of the invention is a N-acyl-homoserine lactonase.

In some embodiments, the ester bond is an ester bond of a homoserine lactone ring.

In some embodiments, homoserine lactone is an acylated homoserine lactone.

In some embodiments, the activity comprises or is quorum quenching. In some embodiments, the isolated polypeptide of the invention is characterized by being capable of quorum-quenching of N-acyl-homoserine lactone. In some embodiments, quorum-quenching comprises N-acyl-homoserine lactone hydrolysis.

As used herein, the term “quorum quenching” refers to inhibition or reduction of bacterial communication via quorum sensing. In some embodiments, quorum quenching further comprises gene expression attenuation.

In some embodiments, the isolated polypeptide is characterized by being stable at temperature of at least 0.1° C., 1° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., or any value and range therebetween. Each possibility represents a separate embodiment of the invention.

In some embodiments, the isolated polypeptide is characterized by being stable at temperature ranging from −20 to 85° C., −10 to 85° C., 0.1 to 85° C., 5 to 85° C., 10 to 90° C., 10 to 80° C., 15 to 80° C., or 5 to 80° C. Each possibility represents a separate embodiment of the invention.

In some embodiments, the isolated polypeptide is characterized by being stable at a pH of at least 4.0, 4.5, 5.0, 5.5, 6.0, 6.5, 7.0, 7.5, 8.0, 8.5, 9.0, 9.5, 10.0, 10.5, 11.0, 11.5, or any value and range therebetween. Each possibility represents a separate embodiment of the invention.

In some embodiments, the isolated polypeptide is characterized by being stable at a pH ranging from 4.5 to 11.0, 5.0 to 11.5, 5.5 to 10.5, 4.5 to 10.0, 5.0 to 9.5, 5.5 to 9.0. Each possibility represents a separate embodiment of the invention.

In some embodiments, the isolated polypeptide is characterized by being stable at a salinity of at least 0.01 M, 0.1 M, 0.3 M, 0.5 M, 0.7 M, 0.9 M, 1.0 M, 1.3 M, 1.5 M, 2.0 M, 2.5 M, 3.0 M, 3.5 M, 4.0 M, 4.5 M, 5.0 M, or any value and range therebetween. Each possibility represents a separate embodiment of the invention.

In some embodiments, the isolated polypeptide is characterized by being stable at a salinity ranging from 0 to 5 M, 0.1 to 4.9 M, 0.5 to 5.5 M, 0.1 to 4 M, 0.3 to 4.8 M, 0.7 to 3.8 M, or 0.5 to 3 M. Each possibility represents a separate embodiment of the invention.

As used herein, the term “stable” refers to the isolated polypeptide of the invention being capable of performing, exhibiting, maintaining, any equivalent thereof, or any combination thereof, the activity as described herein.

In some embodiments, stable is compared to an optimal activity of the isolated polypeptide of the invention as described herein. In some embodiments, stable is compared to an activity of the isolated polypeptide of the invention, achieved under optimal conditions, as described herein.

In some embodiments, stable refers to comprising or maintaining at least 50%, 55%, 60%, 65%, 70%, 75%, 80%, 85%, 90%, 95%, 97%, 99%, or 100% the activity compared to optimal conditions, as disclosed herein, or any value and range therebetween. Each possibility represents a separate embodiment of the invention.

In some embodiments, stable refers to comprising or maintaining 50-80%, 55-85%, 60-90%, 50-99%, 60-95%, 70-99%, 75-97%, 80-95%, 80-100%, or 50-100% the activity compared to optimal conditions, as disclosed herein. Each possibility represents a separate embodiment of the invention.

In some embodiments, the present invention is further directed to an analog and/or a chemically modified form (“derivative”) of the isolated polypeptide of the invention, as long as they are capable of excreting the quorum quenching activity attributed to the isolated polypeptide of the invention, as disclosed hereinbelow.

The term “analog” includes any amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention.

In some embodiments, the analog is a functional analog.

As used herein, “a functional analog” refers to any polypeptide analogous to the isolated polypeptide of the invention and characterized by having essentially the same activity as the isolated polypeptide of the invention, as described herein.

In some embodiments, an activity comprises hydrolysis of a C4 or C8-oxo homoserine lactone, such as acylated homoserine lactone (AHL).

In some embodiments, hydrolysis of a C4 or C8-oxo AHL, as disclosed herein, give rise to a linear AHL.

In some embodiments, essentially the same is at least 90%, 93%, 95%, 96%, 97%, 98%, 99%, or 100% compared to the polypeptide of the invention, or any value and range therebetween. Each possibility represents a separate embodiment of the invention. In some embodiments, essentially the same is 90% to 95%, 92% to 98%, 90% to 99%, 90% to 100%, 94% to 99%, or 95% to 100%, compared to the polypeptide of the invention. Each possibility represents a separate embodiment of the invention.

The phrase “conservative substitution” also includes the use of a chemically derivatized residue in place of a non-derivatized residue provided that such peptide displays the requisite function of modulating the immune system's innate response as specified herein.

The term “derivative” or “chemical derivative” includes any chemical derivative of the polypeptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

The term “derived from” or “corresponding to” refers to construction of an amino acid sequence based on the knowledge of a sequence using any one of the suitable means known to one skilled in the art, e.g., chemical synthesis in accordance with standard protocols in the art.

In addition, a polypeptide derivative can differ from the sequence of the isolated polypeptide of the invention by chemical modifications including, but are not limited to, terminal-NH2 acylation, acetylation, or thioglycolic acid amidation, and by terminal-carboxlyamidation, e.g., with ammonia, methylamine, and the like. Peptides can be either linear, cyclic, or branched and the like, which conformations can be achieved using methods well known in the art.

The polypeptide derivatives according to the principles of the present invention can also include side chain bond modifications, including but not limited to —CH2-NH—, —CH2-S—, —CH2-S═O, OC—NH—, —CH2-O—, —CH2-CH2-, S═C—NH—, and —CH═CH—, and backbone modifications such as modified peptide bonds. Peptide bonds (—CO—NH—) within the peptide can be substituted, for example, by N-methylated bonds (—N(CH3)-CO—); ester bonds (—C(R)H—C-0-0-C(R)H—N); ketomethylene bonds (—CO—CH2-); a-aza bonds (—NH—N(R)—CO—), wherein R is any alkyl group, e.g., methyl; carba bonds (—CH2-NH—); hydroxyethylene bonds (—CH(OH)—CH2-); thioamide bonds (—CS—NH); olefmic double bonds (—CH—CH—); and peptide derivatives (—N(R)—CH2-CO—), wherein R is the “normal” sidechain, naturally presented on the carbon atom. These modifications can occur at one or more of the bonds along the peptide chain and even at several (e.g., 2-3) at the same time.

In some embodiments, the polypeptide derivative contains non-natural amino acids. Examples of non-natural amino acids include, but are not limited to, sarcosine (Sar), norleucine, ornithine, citrulline, diaminobutyric acid, homoserine, isopropyl Lys, 3-(2-naphtyl)-Ala, nicotinyl Lys, amino isobutyric acid, and 3-(3-pyridyl-Ala).

In some embodiments, the polypeptide derivative contains other derivatized amino acid residues. Examples of derivatized amino acid residues include, but are not limited to, methylated amino acids, N-benzylated amino acids, O-benzylated amino acids, N-acetylated amino acids, O-acetylated amino acids, carbobenzoxy-substituted amino acids and the like. Specific examples include, but are not limited to, methyl-Ala (Me Ala), MeTyr, MeArg, MeGlu, MeVal, MeHis, N-acetyl-Lys, O-acetyl-Lys, carbobenzoxy-Lys, Tyr-O-Benzyl, Glu-O-Benzyl, Benzyl-His, Arg-Tosyl, t-butylglycine, t-butylalanine, phenylglycine, cyclohexylalanine, and the like.

In some embodiments, the invention is further directed to a polypeptide analog, which contains one or more D-isomer forms of the amino acids. Production of retro-inverso D-amino acid peptides where at least one amino acid, and perhaps all amino acids are D-amino acids is well known in the art. When all of the amino acids in the peptide are D-amino acids, and the N- and C-terminals of the molecule are reversed, the result is a molecule having the same structural groups being at the same positions as in the L-amino acid form of the molecule. However, the molecule is more stable to proteolytic degradation and is therefore useful in many of the applications recited herein. Diastereomeric peptides may be highly advantageous over all L- or all D-amino acid peptides having the same amino acid sequence because of their higher water solubility, lower immunogenicity, and lower susceptibility to proteolytic degradation. The term “diastereomeric peptide” as used herein refers to a peptide comprising both L-amino acid residues and D-amino acid residues. The number and position of D-amino acid residues in a diastereomeric peptide of the preset invention may be variable so long as the peptide is capable of displaying the requisite function, e.g., anti-inflammatory activity, as specified herein.

The term “analog” and “derivative” are used herein interchangeably.

The isolated polypeptide of the invention may be synthesized or prepared by techniques well known in the art. The isolated polypeptide can be synthesized by a solid phase peptide synthesis method of Merrifield (see J. Am. Chem. Soc, 85:2149, 1964). Alternatively, the polypeptide of the present invention can be synthesized using standard solution methods well known in the art (see, for example, Bodanszky, M., Principles of Peptide Synthesis, Springer-Verlag, 1984) or by any other method known in the art for peptide synthesis.

In general, the aforementioned methods comprise sequential addition of one or more amino acids or suitably protected amino acids to a growing peptide chain bound to a suitable resin.

Normally, either the amino or carboxyl group of the first amino acid is protected by a suitable protecting group. The protected or derivatized amino acid can then be either attached to an inert solid support (resin) or utilized in solution by adding the next amino acid in the sequence having the complimentary (amino or carboxyl) group suitably protected, under conditions conductive for forming the amide linkage. The protecting group is then removed from this newly added amino acid residue and the next amino acid (suitably protected) is added, and so forth. After all the desired amino acids have been linked in the proper sequence, any remaining protecting groups are removed sequentially or concurrently, and the peptide chain, if synthesized by the solid phase method, is cleaved from the solid support to afford the final peptide.

In the solid phase peptide synthesis method, the alpha-amino group of the amino acid is protected by an acid or base sensitive group. Such protecting groups should have the properties of being stable to the conditions of peptide linkage formation, while being readily removable without destruction of the growing peptide chain. Suitable protecting groups are t-butyloxycarbonyl (BOC), benzyloxycarbonyl (Cbz), biphenylisopropyloxycarbonyl, t-amyloxycarbonyl, isobornyloxycarbonyl, (alpha, alpha)-dimethyl-3,5dimethoxybenzyloxycarbonyl, o-nitrophenylsulfenyl, 2-cyano-t-butyloxycarbonyl, 9-fluorenylmethyloxycarbonyl (FMOC) and the like.

In the solid phase peptide synthesis method, the C-terminal amino acid is attached to a suitable solid support. Suitable solid supports useful for the above synthesis are those materials, which are inert to the reagents and reaction conditions of the stepwise condensation-deprotection reactions, as well as being insoluble in the solvent media used. Suitable solid supports are chloromethylpolystyrene-divinylbenzene polymer, hydroxymethyl-polystyrene-divinylbenzene polymer, and the like. The coupling reaction is accomplished in a solvent such as ethanol, acetonitrile, N,N-dimethylformamide (DMF), and the like. The coupling of successive protected amino acids can be carried out in an automatic polypeptide synthesizer as is well known in the art.

The isolated polypeptide of the invention may alternatively be synthesized such that one or more of the bonds, which link the amino acid residues of the polypeptide are non-peptide bonds. These alternative non-peptide bonds include, but are not limited to, imino, ester, hydrazide, semicarbazide, and azo bonds, which can be formed by reactions well known to skilled in the art.

In some embodiments, recombinant protein techniques are used to generate the polypeptide of the invention. In some embodiments, recombinant protein techniques are used for generation of relatively long peptides (e.g., longer than 18-25 amino acids). In some embodiments, recombinant protein techniques are used for the generation of large amounts of the polypeptide of the invention. In some embodiments, recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al, (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Any one of the isolated polypeptide of the present invention, an analog thereof, and a derivative thereof, produced by recombinant techniques can be purified so that the polypeptide will be substantially pure when administered to a subject. The term “substantially pure” refers to a compound, e.g., a polypeptide, which has been separated from components, which naturally accompany it.

Typically, a polypeptide is substantially pure when at least 50%, at least 75%, at least 90%, and at least 99% of the total material (by volume, by wet or dry weight, or by mole percent or mole fraction) in a sample is the polypeptide of interest, or any value and range therebetween. Each possibility represents a separate embodiment of the invention. Purity can be measured by any appropriate method, e.g., in the case of peptides by HPLC analysis.

### Nucleic Acids

According to some embodiments, there is provided a polynucleotide encoding the isolated polypeptide of the invention.

In some embodiments, the polynucleotide comprises a nucleic acid sequence being codon optimized for expression in a cell.

In some embodiments, the cell comprises a bacterial cell or a fungal cell.

In some embodiments, polynucleotide comprises the nucleic acid sequence:

(SEQ ID NO: 4)
CACGAGCACGTGCTGGTTACCAGCGCGGGTATCCAGTACGTGTATCCGG
AGTTCATCGACCGTGAAGGCACCATTGCGCGTGCGGTTGCGGATCTGAA
GACCGCGTATAGCGAAGGTCTGCGTACCATCGTGGACGTTACCACCATT
GACCTGGGCCGTGACATCCGTATGCTGGAGCAAGTGAGCCGTGAAAGCG
GTATTAACATCATTTGCGCGACCGGCACCTGGCGTGACATCCCGCGTGT
GTTTTGGAGCGCGAGCCCGGATATGGTTGCGCCGCTGTACATCCGTGAG
ATTGAGGAAGGTATCGAAGGCACCGGCATTAAGGCGGCGATCATTAAAG
TTGCGAACGATGTGGGTGGCGTTACCCCGGAGGGCGAAATCATTCTGCG
TGCGGCGGCGCGTGCGCAGAAGGCGACCGGTGTGCCGATTAGCACCCAT
ACCTGGGCGCCGGAGCGTGTGGGTGAACAGCAAGTTCGTATCTTCGAGG
ACGAAGGTGTTGATCTGAACCGTGTGTACGTTGGCCACAGCAACGACAC
CACCGATACCGAGTATCTGATTGGTCTGCTGGAAAAAGGTGTGTGGATC
GGCCTGGACCGTTATCCGGGTCGTCAGACCGAGCACACCCCGGATTGGA
TCGGCCGTACCGAAACCGCGAAGAAACTGATTGACGCGGGTTATGGCCA
CCGTATCATGCTGGGTCACGATTGGAGCGTTACCCTGAGCATTGCGAGC
AAGGAGATGCAGGAACAACGTCTGAAATACAACCCGGACGGTTACCTGT
TCATCACCCGTAACGTGGTTCCGAAGCTGAAAGAGCTGGGCGCGACCGA
GGAAGAC.

In some embodiments, the polynucleotide comprises the nucleic acid sequence:

(SEQ ID NO: 2)
ATGGCGACCATTAACAGCGTTCTGGGTCCGCTGGACACCGCGGACATCG
GCTACACCCTGAGCCACGAGCACGTGCTGGTTACCAGCGCGGGTATCCA
GTACGTGTATCCGGAGTTCATCGACCGTGAAGGCACCATTGCGCGTGCG
GTTGCGGATCTGAAGACCGCGTATAGCGAAGGTCTGCGTACCATCGTGG
ACGTTACCACCATTGACCTGGGCCGTGACATCCGTATGCTGGAGCAAGT
GAGCCGTGAAAGCGGTATTAACATCATTTGCGCGACCGGCACCTGGCGT
GACATCCCGCGTGTGTTTTGGAGCGCGAGCCCGGATATGGTTGCGCCGC
TGTACATCCGTGAGATTGAGGAAGGTATCGAAGGCACCGGCATTAAGGC
GGCGATCATTAAAGTTGCGAACGATGTGGGTGGCGTTACCCCGGAGGGC
GAAATCATTCTGCGTGCGGCGGCGCGTGCGCAGAAGGCGACCGGTGTGC
CGATTAGCACCCATACCTGGGCGCCGGAGCGTGTGGGTGAACAGCAAGT
TCGTATCTTCGAGGACGAAGGTGTTGATCTGAACCGTGTGTACGTTGGC
CACAGCAACGACACCACCGATACCGAGTATCTGATTGGTCTGCTGGAAA
AAGGTGTGTGGATCGGCCTGGACCGTTATCCGGGTCGTCAGACCGAGCA
CACCCCGGATTGGATCGGCCGTACCGAAACCGCGAAGAAACTGATTGAC
GCGGGTTATGGCCACCGTATCATGCTGGGTCACGATTGGAGCGTTACCC
TGAGCATTGCGAGCAAGGAGATGCAGGAACAACGTCTGAAATACAACCC
GGACGGTTACCTGTTCATCACCCGTAACGTGGTTCCGAAGCTGAAAGAG
CTGGGCGCGACCGAGGAAGACATCCAGAACATTTTTGTTAACAACCCGC
GTAACTTCTTTGAAGCGAGC.

In some embodiments, the polynucleotide comprising the nucleic acid sequence set forth in SEQ ID NO: 2 or SEQ ID NO: 4, is codon optimized for expression in a cell, as described herein.

In some embodiments, SEQ ID NO: 2 is codon optimized for expression in a cell, as described herein.

In some embodiments, SEQ ID NO: 4 is codon optimized for expression in a cell, as described herein.

In some embodiments, the polynucleotide is codon optimized for expression in a bacterial cell.

In some embodiments, the polynucleotide is codon optimized for expression in an E. coli cell.

In some embodiments, the polynucleotide comprises a nucleic acid sequence having at least 70%, 80%, 90%, 95%, 97%, 99%, or 100% identity to SEQ ID NO: 2, or any value and range therebetween. Each possibility represents a separate embodiment of the invention.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

According to some embodiments, there is provided an artificial nucleic acid molecule or vector comprising the polynucleotide disclosed herein.

In some embodiments, the artificial nucleic acid molecule or vector comprises a plasmid. In some embodiments, the artificial nucleic acid molecule or vector is an expression vector. In some embodiments, the artificial nucleic acid molecule or vector is for use in heterologous expression of the polynucleotide as disclosed herein in a cell, a tissue, or an organism. In some embodiments, the artificial nucleic acid molecule or vector is for use in producing or the production of isolated polypeptide of the invention in a cell, a tissue, or an organism, a cell culture, or any combination thereof.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3: 1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial nucleic acid molecule or vector comprises a polynucleotide encoding a polypeptide comprising an amino acid sequence as described herein.

### Cells, Extracts, and Compositions

According to some embodiments, there is provided a cell comprising any one of: (a) the isolated polypeptide of the invention; (b) the polynucleotide encoding same; (c) the artificial nucleic acid molecule or vector comprising the polynucleotide; and (d) any combination of (a) to (c).

In some embodiments, the cell is or comprises a transgenic cell, a transformed cell, or a transfected cell.

In some embodiments, the cell is selected from: a unicellular organism, a cell of a multicellular organism, or a cell in a culture.

In some embodiments, the cell comprises a plant cell. In some embodiments, the cell comprises an animal cell. In some embodiments, the cell comprises a mammalian cell. In one embodiment, the cell comprises a macrophage.

In some embodiments, the cell is a bacterial cell.

In some embodiments, the cell is an E. coli cell.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, a unicellular organism comprises a fungus or a bacterium.

In some embodiments, the bacterium is Escherichia coli cell.

In some embodiments, the fungus is a yeast cell.

According to some embodiments, there is provided an extract obtained or derived from a cell disclosed herein, or any fraction thereof.

In some embodiments, the extract comprises the isolated polypeptide of the invention, the herein disclosed polynucleotide, the artificial nucleic acid molecule or vector disclosed herein, a protein as disclosed herein, or any combination thereof.

According to some embodiments, there is provided a homogenate, lysate, extract, derived from a cell as disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar or nonpolar solvent), liquid chromatography mass spectrometry, or others.

According to some embodiments, there is provided a composition comprising any one of: (a) the isolated polypeptide of the invention; (b) the herein disclosed polynucleotide; (c) the herein disclosed artificial nucleic acid molecule vector; (d) the herein disclosed cell; (e) the herein disclosed extract; and (f) any combination of (a) to (d). In some embodiments, the composition comprises or further comprises an acceptable carrier.

In some embodiments, the composition of the invention comprises or is characterized by being capable or having an activity of preventing or reducing food spoilage.

In some embodiments, the composition is in the form of a capsule.

In some embodiments, a capsule comprises a nanopeptide capsule, peptide nanotube, or both.

In some embodiments, food spoilage is induced by or involves a bacterium, biofilm produced by same, or both.

In some embodiments, the bacterium comprises Pseudomonas fluorescens.

As used herein, the term “food spoilage” refers to a process wherein food becomes unsuitable for consumption by a subject. In some embodiments, unsuitable comprises unsuitable for ingestion by a subject. In some embodiments, unsuitable to be understood as increasing the risk of developing an infection including any disorder or a symptom associated therewith, in case of being ingested by a subject.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

According to some embodiments, there is provided an article comprising the isolated polypeptide of the invention.

According to some embodiments, an article (e.g., an article-of-manufacturing) comprises a substrate incorporating in and/or on at least a portion thereof at least one of: an isolated polypeptide of the invention or a composition comprising same.

In some embodiments, the isolated polypeptide is bound to a surface of the article.

In some embodiments, bound comprises covalently bound.

In some embodiments, the isolated polypeptide is immobilized to a surface of an article.

As used herein, the terms “bound” and “immobilized” are interchangeable.

In some embodiments, the isolated polypeptide disclosed herein, is bound, or immobilized to a surface, e.g., of an article, via the N′ terminus of the isolated polypeptide.

The N′ terminus of the isolated polypeptide comprises the first amino acid residue of the isolated polypeptide. In some embodiments, the isolated polypeptide disclosed herein, is bound or immobilized to a surface, e.g., of an article, via the first amino acid residue at the N′ terminus.

In some embodiments, the article is configured to storing or transferring a liquid.

The article can be any article which can benefit from the quorum quenching and/or anti-biofilm formation activities of the isolated polypeptide disclosed herein.

Non-limiting examples of articles include, but are not limited to, medical devices, organic waste processing device, fluidic device, an agricultural device, a package, a sealing article, a fuel container, a water and cooling system device and a construction element.

Non-limiting examples of devices which can incorporate at least one of: the isolated polypeptide of the invention or a composition comprising same, as described herein, beneficially, include tubing, pumps, drain or waste pipes, screw plates, and the like.

Non-limiting example of an article include but is not limited to an element used in water treatment systems (such as for containing and/or transporting and/or treating aqueous media or water), devices, containers, filters, tubes, solutions and gases and the likes.

Non-limiting example of an article include but is not limited to an element in organic waste treatment systems (such as for containing and/or disposing and/or transporting and/or treating organic waste), devices, containers, filters, tubes, solutions and gases and the likes.

### Methods of Use

According to some embodiments, there is provided a method for inhibiting or reducing a formation of load of organic-based contaminant in a composition.

In some embodiments, the composition is susceptible of the formation of an organic based contaminant.

According to some embodiments, there is provided a method for inhibiting or reducing a formation of load of organic-based contaminant on or within an article.

In some embodiments, the method comprises contacting the composition with an effective amount of the isolated polypeptide of the invention.

In some embodiments, the method comprises coating or incorporating a composition comprising the isolated polypeptide of the invention to an article.

In some embodiments, incorporating comprises binding the isolated polypeptide to a surface of the article.

In some embodiments, binding comprises covalently binding.

In some embodiments, the article is configured to storing or transferring a liquid. In some embodiments, the article is configured to allow liquid flow therethrough.

In some embodiments, the liquid comprises or consists of water. In some embodiments, the liquid is a water-based liquid. In some embodiments, the liquid comprises wastewater or a treated product or intermediate thereof. In some embodiments, the liquid comprises milk, a precursor thereof, or a product or an intermediate thereof.

As used herein, the term “water-based” refers to any liquid wherein water is the main solvent (e.g., at least 50% by weight, by volume, or both, of the solvent partiality in the liquid is water).

In some embodiments, the article comprises a tubing, a capsule, a container, a vessel, a chamber, a reservoir, or any combination thereof.

According to some embodiments, there is provided a composition obtained or derived from the method of the invention.

In some embodiments, the composition is an edible or a comestible composition.

In some embodiments, the composition is or comprises a food product.

In some embodiments, the composition is or comprises a dairy product. In some embodiments, the composition is or comprises a milk product. In some embodiments, the composition is or comprises milk.

In some embodiments, a composition as disclosed herein, being obtained or derived from the method of the invention is characterized by having a weight per weight or molar per molar ratio of cyclic homoserine lactone to linear homoserine lactone being lower than 1. In some embodiments, a composition as disclosed herein, being obtained or derived from the method of the invention is characterized by having a greater amount or concentration of linear homoserine lactone compared to the amount or concentration of cyclic homoserine lactone. In some embodiments, a composition as disclosed herein, being obtained or derived from the method of the invention is characterized by having a greater amount or concentration of linear homoserine lactone compared to the amount or concentration of linear homoserine lactone in a control food composition not being treated according to the method of the invention. In some embodiments, a composition as disclosed herein, being obtained or derived from the method of the invention is characterized by having a lower amount or concentration of cyclic homoserine lactone compared to the amount or concentration of cyclic homoserine lactone in a control food composition not being treated according to the method of the invention.

In some embodiments, the food product is or comprises a meat product.

In some embodiments, the organic-based contaminant comprises biofilm.

In some embodiments, the biofilm is produced by a Pseudomonas bacterium. In some embodiments, the organic-based contaminant comprises Pseudomonas bacteria. In some embodiments, the organic-based contaminant comprises biofilm and at least one cell of a Pseudomonas bacterium. In some embodiments, the organic-based contaminant comprises planktonic cells of Pseudomonas and biofilm produced by same. In some embodiments, Pseudomonas is or comprises Pseudomonas fluorescens.

In some embodiments, the composition of the invention is characterized by and/or utilized in a pH ranging from 2 to 11, 2 to 10, 2 to 9, 2 to 8, 2 to 7, 3 to 11, 3 to 10, 3 to 9, 3 to 8, 3 to 7, 4 to 11, 4 to 10, 4 to 9, 4 to 8, 4 to 7, 5 to 11, 5 to 10, 5 to 9, 5 to 8, 5 to 7, 6 to 11, 6 to 10, 6 to 9, 6 to 8, 6 to 7.5, 6 to 7. Each possibility represents a separate embodiment. In some embodiments, the composition of the invention is utilized and/or kept in a temperature ranging from −20 to 90° C., −20 to 80° C., −10 to 90° C., −10 to 80° C., 0.1 to 90° C., or 0.1 to 80° C.

According to some embodiments, there is provided a composition an edible or a comestible composition.

In some embodiments, the composition is or comprises a food product.

In some embodiments, the composition is or comprises a dairy product. In some embodiments, the composition is or comprises a milk product. In some embodiments, the composition is or comprises milk. In some embodiments, the food product is or comprises a meat product.

In some embodiments, the composition is characterized by having a weight per weight or molar per molar ratio of cyclic homoserine lactone to linear homoserine lactone being lower than 1. In some embodiments, the composition is characterized by having a greater amount or concentration of linear homoserine lactone compared to the amount or concentration of cyclic homoserine lactone.

### General

As used herein the term “about” refers to +10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., cd. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization-A Laboratory Course Manual” CSHL Press (1996); all of which are incorporated by reference. Other general references are provided throughout this document.

### Materials and Methods

### Sequence Identification, Alignment and Phylogenetic Tree

The sequence of a SsoPox, an AHL lactonase from the PTE-Like-Lactonase (PLL) family from S. solfataricus (pdb|2VC5|A; Elias et al., 2008, JMB) was used as a probe to search for PTE-Like-Lactonases within in-house assemblies of 244 metagenomes from the Tara Oceans project. All predicted proteins from the Tara assemblies were aligned against the probe sequence (blastp v2.3.0, e-value threshold 1e-50). The best 250 hits were clustered using usearch to create a non-redundant set of 144 potential PTE-Like-Lactonases. Next, 21 sequences with 30-90% identity were aligned with Muscle algorithm, in the MEGA 7 software (MEGA7: Molecular Evolutionary Genetics Analysis version 7.0 for bigger datasets; Kumar, Stecher, and Tamura 2015). An alignment picture was created with Javlview. A phylogenetic tree of PLL homologs was inferred by the Neighbor-Joining method using MEGA X software. PTE, Phosphotriesterase from Pseudomonas diminuta now known as Brevundimonas diminuta, was used an outgroup to allow phylogeny to be rooted, as it share about 30% identity with PLLs but differ in specificity. The bootstrap resample test was performed 100 times to confirm the tree's reliability.

### Evaluation of Completeness and Taxonomy of the Metagenomics Assembled Genome (MAG) GCA_009392485.1.

The inventors downloaded the protein and genomics sequences for the MAG from the GenBank ftp site. Completeness and contamination were evaluated using checkm v1.1.3 with the lineage_wf workflow. Taxonomy was assigned using gtdbtk v1.4.1, database release 95, with the classify_wf workflow.

### Structure Modeling

A 3D structural model of moLRP was generated by submitting its amino acid sequence to SWISS-MODEL online sever (https://swissmodel.expasy.org/), an automated software that calculates models on the basis of known structural templates and sequence-structure alignments. The solved structure of the thermostable quorum-quenching lactonase from Geobacillus kaustophilus GKL (pdb 3ojg, sharing 35% identity with moLRP) was used as template. Next, the structural model was aligned to the structure of catalytically inactive D266N mutant of this enzyme to a resolution of 1.6 Å with a bound substrate, C4-HSL (pdb 3ojg) by PyMOL Molecular Graphics System, Version 1.2r3pre, Schrödinger, LLC.

### Gene Synthesis, Cloning, Expression, and Purification

The E. coli codon optimized synthetic gene encoding two putative AHL-Lactonase marine homolog, named moLRPs (marine originated Lactonase Related Proteins), were ordered from Genscript cloned into pET-21a (+) vector. The plasmids were elctro-transformed into E. coli-BL21 (DE3) cells. The functional expression and activity of the newly identified marine enzymes was tested in the presence of different divalent metals and were tested by transforming the plasmids into BL21 (DE3) E. coli cells, as previously described. Enzymatic activity in the crude lysates was measured with lactone substrate chromogenic thio-buthyl-γ-butyric-lactone (TBBL), using 5,5′-dithiobis 2-nitrobenzoic acid (DTNB) indicator for free thiol (Ellman's reagent) as described. A previously characterized, AHL lactonase from the PLL family, PPH (Parathion hydrolase from Mycobacterium tuberculosis was also expressed and purified as previously described). For large-scale production, a single colony of E. coli BL21 (DE3) cells freshly transformed with pET-21-moLRP or pMAL-c2x-PPH was inoculated in LB medium (5 mL) containing 50 μg/mL kanamycin or 100 μg/mL ampicillin, respectively, and 0.1 mM ZnCl₂ (pET21-moLRP) or 0.1 mM MnCl₂ (pMAL-c2x-PPH), and grown overnight, at 37° C. The resulting cultures were added to 500 mL of the same medium and grown, shaking at 30° C. for PPH and 37° C. for moLRP. At OD₆₀₀=0.5-0.7, over-expression was induced by adding 0.4 mM Isopropyl β-d-1-thiogalactopyranoside (IPTG), followed by an overnight growth at 30° C. Cells were harvested by centrifugation and suspended in lysis buffer (100 mM Tris-HCl pH 8, 100 mM NaCl, histidine-tagged protease inhibitor Cocktail (Sigma) diluted 1:500, and 100 μM ZnCl₂ or MnCl₂). Subsequent steps were all performed at 4° C. Ni-NTA beads (PureCube, Cube biotech) in gravity columns were equilibrated with filtered column buffer (100 mM Tris-HCl pH 8, 100 mM NaCl, and 100 μM ZnCl₂ or MnCl₂). After centrifugation, supernatant of cells expressing moLRP were passed through the gravity column containing Ni-NTA Agarose. Supernatant of cells expressing PPH was passed through an amylose column (MBPTrap™ HP, GE Healthcare), suited for the AKTA fast protein liquid chromatography (FPLC) system. Proteins were eluted, with column buffer supplemented with 300 mM Imidazole (for moLRP) or 10 mM maltose (for PPH), and the enzymatic activity of the collected fractions were analyzed with TBBL. Fractions with highest activity were pooled and dialyzed with activity buffer. Purity of the proteins was analyzed by 15% SDS-PAGE for moLRP and 12% SDS-PAGE for PPH. Proteins were then stored at 4° C.

### Enzyme Activity Measurements

Enzymatic hydrolysis by moLRP was analyzed by monitoring absorbance changes in 200 μL reaction volumes incubated in 96-well plates, using a microtiter plate reader (BioTek Synergy H1). The chromogenic lactonase assay used for the biochemical characterization was based on thio-buthyl-γ-butyric-lactone (TBBL) and 5,5-dithiobis 2-nitrobenzoic acid (DTNB), monitoring wavelength 412 nm, as described in. The extinction coefficient for the 0.5 cm pathway is 7,000 OD/M, and final organic solvent content was 1% acetonitrile. Activity was measured with enzyme concentration of 0.3 μM for moLRP in its activity buffer, 50 mM Tris-HCl pH 8, 100 mM NaCl, and 100 μM ZnCl₂. PPH activity with AHLs was previously described. The hydrolysis of N-acyl homoserine lactones was monitored by following the appearance of the carboxylic acid products using a pH indicator, cresol purple, as described previously. The tested AHLs purchased from Sigma-Aldrich were: N-butyryl-L-homoserine lactone (C4-HSL), N-heptanoyl-L-homoserine lactone (C7-HSL), N-(3-oxohexanoyl)-L-homoserine lactone (3-Oxo-C6-HSL), N-octanoyl-L-homoserine lactone (C8-HSL), N-(3-oxooctanoyl)-DL-homoserine lactone (3-Oxo-C8-HSL) and N-oxodeconyl-DL-homoserine lactone (C10-HSL). Enzyme initial velocities (V₀) were corrected for the background rate of spontaneous hydrolysis in the absence of enzyme. Kinetic parameters were obtained by fitting initial rates directly to the Michaelis-Menten equation [V₀=k_{cat}[E]₀[S]₀/([S]₀+K_{M})] with GraphPad. Error ranges relate to the standard deviation of the data obtained from at least three independent measurements.

### Enzyme Optimal Temperature and Thermal Inactivation Assay

To determine the optimal temperature for activity of purified moLRP (0.025 μM) and PPH (1 μM), enzymes were incubated with (0.1 mM) and DTNB (0.5 mM) in activity buffer, at various temperatures (5-95° C.). Samples collected at time 0 and after 15 minutes, were analyzed at 412 nm. The control sample was prepared under the same conditions but without the enzyme, and their values were subtracted from each corresponding test sample containing the enzyme. Readings of pre-incubation samples were subtracted from the reading of post incubation samples. 100% activity was defined as the activity at 35° C. for moLRP and 45° C. for PPH. Each treatment was replicated three times. For thermal inactivation, 0.15 μM moLRP or 1 μM PPH was incubated at temperatures ranging from 5° C. to 95° C., for 45 min. Enzyme activity was then determined with 0.1 mM TBBL and 0.5 mM DTNB at 25° C. Spontaneous hydrolysis in samples without the enzyme were used as control. The residual activity of moLRP at different temperatures is shown as the percentage of the highest activity measured at 80° C., while the residual activity of PPH is displayed as the percentage of highest activity measured in 15° C. in this study.

### Optimal pH for Enzyme Activity and Enzyme Stability at Different pH Values

To test the optimal pH for enzymes activity, purified moLRP (0.3 μM) and PPH (1.75 M) were diluted in buffers with pH values ranging from 3.5 to 11 (100 mM acetate buffer for pH 3.5-5.5, phosphate buffer for pH 5.5-8.0, Tris-HCl buffer for pH 8.0-9.0, and carbonate-bicarbonate buffer for pH 9.0-11.0). Enzymes activity was measured with 0.1 mM TBBL and 0.5 mM DTNB at the corresponding pH level, at 25° C. Spontaneous hydrolysis of TBBL in the activity buffer at each pH range was subtracted from corresponding test samples. Each treatment was repeated three times. The relative activity is defined as the percentage from highest activity of moLRP and PPH at their optimal pH level, pH 8 and pH 9, respectively. For enzyme stability in different pH ranges, purified moLRP (2.5 μM) was incubated for one hour at 4° C., in buffers with pH ranging from 3.5 to 11.0. Enzyme activity was then measured by addition of 0.1 mM TBBL and 0.5 DTNB in activity buffer with the optimal pH for each enzyme, at 25° C. Spontaneous hydrolysis of TBBL in the activity buffer was subtracted from the corresponding test samples. Each treatment was repeated three times. Residual activity of moLRP and PPH was determined compared to highest activity measured at pH 8 or 9, respectively.

### Enzyme Activity and Stability in Different Salinity Levels

The activity of moLRP (0.3 μM) or PPH (2 μM) in activity buffer (100 mM Tris-HCl, pH 8 or 9, 100 μM ZnCl₂ or MnCl₂, respectively) was tested in different salinity concentration from 0 M to 5 M. Enzyme activity was measured as described above. Each treatment was repeated three times. For each protein, the relative activity was compared to its optimal salinity level for activity. For enzyme stability in different salinity level, moLRP (0.3 μM) and PPH (2 μM) were incubated in buffers with salinity ranging from 0 M to 5 M for one hour at 4° C. Enzyme activity was then measured with optimal pH and salinity for each enzyme 25° C. Each treatment was repeated three times. Residual activity of moLRP was determined in comparison to the enzyme activity measured at 5 M salinity, whereas residual activity of PPH was determined compared to enzyme activity recorded in 2 M.

### Bacterial Strains and Growth Medium

Pseudomonas fluorescens strain used was Migula ATCC 948. Bacterial colonies that presented high proteolytic activity on milk solid medium were sequenced and verified as P. fluorescens using 16S rRNA primers. The bacteria were grown at 28° C., under 170 rpm in LB Broth. Sterilized 10% Skim milk (121° C. for 15 min, Sigma Switzerland) was used as test medium for bacterial activity.

Growing Cultures of P. fluorescens in Skim Milk in the Presence of Purified Enzymes

Over-night cultures (10 μl) of P. fluorescens at OD₆₀₀=0.7 grown in LB, were inoculated in 2 ml skim milk medium with or without the addition of 1 μM purified enzymes. Skim milk alone was used as a negative control. Cultures were grown at 28° C. for 4 hours at 170 rpm, and then incubated without shaking for 7 days. Pictures of cultures were taken after 7 days. Each treatment was tested in three biological replicates.

### Purified Enzyme Residual Activity Following Incubation in Bacterial Culture

Overnight cultures of P. fluorescens (OD₆₀₀=1), were diluted (1:3 ratio) in LB medium alone or containing 1 μM purified enzymes, and incubated at 28° C., 170 rpm. Each treatment was performed in three biological replicates. Samples of 200 μl were taken at different time points from inoculation, centrifuged at 10,000 RPM at 4° C. for 5 min. Next, bacterial supernatant (30 μl in duplicates) was used to test enzyme activity with 0.05 mM TBBL and DTNB (0.5 mM). Activity of supernatants from cultures without exogenically added enzyme was subtracted from the activity of supernatants from cultures with enzymes addition. Residual activity of each enzyme was calculated compared to the 100% activity in buffer.

Proteolytic Activity of P. fluorescens Supernatants

Proteolytic activity was determined using azocasein assay adjusted to skim milk medium as previously established. Briefly, overnight cultures of P. fluorescens in LB (OD₆₀₀=1) were diluted (1:100) in 10% skim milk medium containing 1 μM purified enzyme. After 24 h growth at 28° C., cultures were centrifuged at 8,000 g, for 10 min at 4° C. 150 μl of culture supernatant from each sample was incubated with 250 μL of 2% (w/v) azocasein. The mixture was incubated at 30° C., 300 rpm overnight. Following the addition of 1.2 mL of 10% (w/v) trichloroacetic acid (TCA) to stop the reaction, at room temperature for 15 min, the samples were centrifuged at 4° C., 15,000 g. Next, 600 μL of supernatants were removed and added to 750 μL of 1 M NaOH. Finally, the exo-proteolytic activity of P. fluorescens supernatants was quantified at OD₄₄₀. Skim milk media and skim milk with P. fluorescens were used as controls. Each treatment had three biological replicates. Each sample was used for three independent replicates. Statistical significance according to one way ANOVA.

Biofilm Inhibition of P. fluorescens

To measure the effect of QQ lactonases on biofilm formation, over-night cultures of P. fluorescens in LB (OD₆₀₀=2) were diluted (1:100) in Tryptic Soy Broth (TSB) medium with 0.2% glucose medium, supplemented with final concentration of 1 μM purified PPH or moLRP (by adding 30 μL purified enzyme into to 3 mL TSB medium). Each enzyme's activity buffer was used as a negative control. After 4 h incubation at 28° C., 170 RPM, 200 μL of cultures were dispensed into a sterile U-bottom 96-well tissue culture plate (JET BIOFIL, China), and further incubated for 24 h without shaking. Afterwards, the planktonic cells were removed, and the plates were rinsed three times with double distilled water. Excess moisture was removed by tapping the microplates on sterile napkins, and the plates were air-dried for 15 min. Biofilm formation was quantified using the crystal violet assay, as described before. The absorbance at 590 nm was measured in F-Bottom 96-well microplate (Greiner bio-one, Germany). The presented data is an average from three biological repeats, each having four technical repeats, normalized to the buffer-negative control. Statistical significance was analyzed with one-way ANOVA.

### Particle Velocity Distributions Analysis by LUMiFuge

To quantify the inhibitory effect of purified moLRP on milk proteolysis and sedimentation in the presence of Pseudomonas florescence, the inventors used LUMisizer 6110 (LUM GmbH, Germany), an analytical dispersion analyzer. The instrument records NIR (near infrared) light transmission during centrifugation over the total length of a cell containing the suspension. Based on the light reaching the sensor it prepares a “transmission profile”, enabling characterization of the stability of the emulsion or suspension. High concentrations of particles yield low light transmission, whereas low concentrations of particles (after milk proteolysis and sedimentation or “creaming”) yield high transmissions. The method was used in colloidal analysis for water clarification and to analyze the stability of milk. For this, 2.5 ml of over-night bacterial cultures of Pseudomonas fluorescents (OD₆₀₀=1), diluted in 10% skim milk with or without 1 μM purified moLRP, at 28° C. for 4-6 hours, 170 rpm. As a negative control 10% skim milk without bacteria was used. Next, 400 μL of each of the three treatments were inserted into LUMsizer sample cells [LUM2 mm, PC, rectangular synthetic cell (110-131xx)], in three replicates. Measurements were performed every 150 sec at a mean relative acceleration of 5.2 g for 24 hours at 28° C. The inventors used the integrated light transmission of the upper 20% of each test tube against time. Values were downloaded using a SepView 6.4 software (LUM GmbH, Germany) and three replicates were averaged.

### Example 1

Identification of Putative AHL Lactonases from the PLL Family within Marine Metagenomics Samples

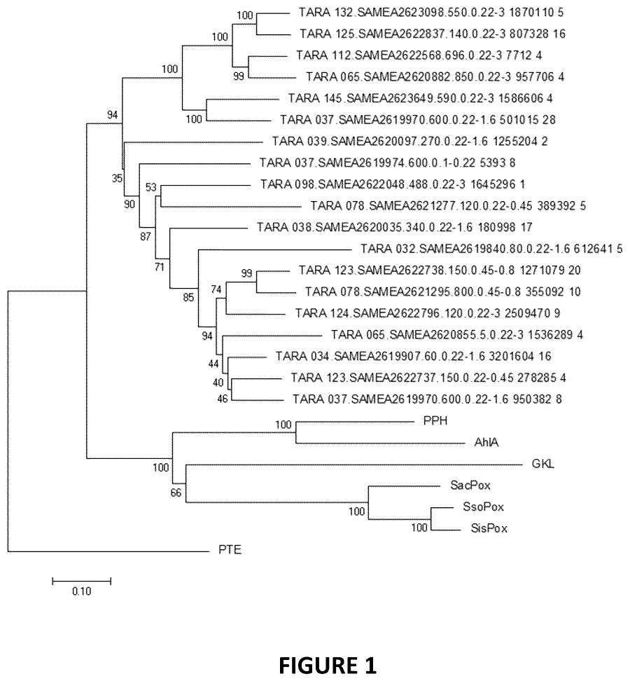

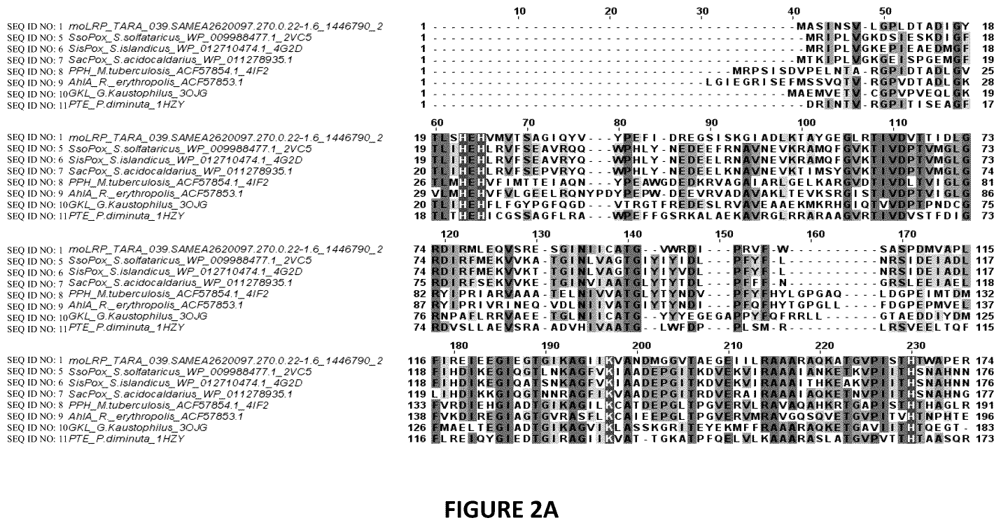

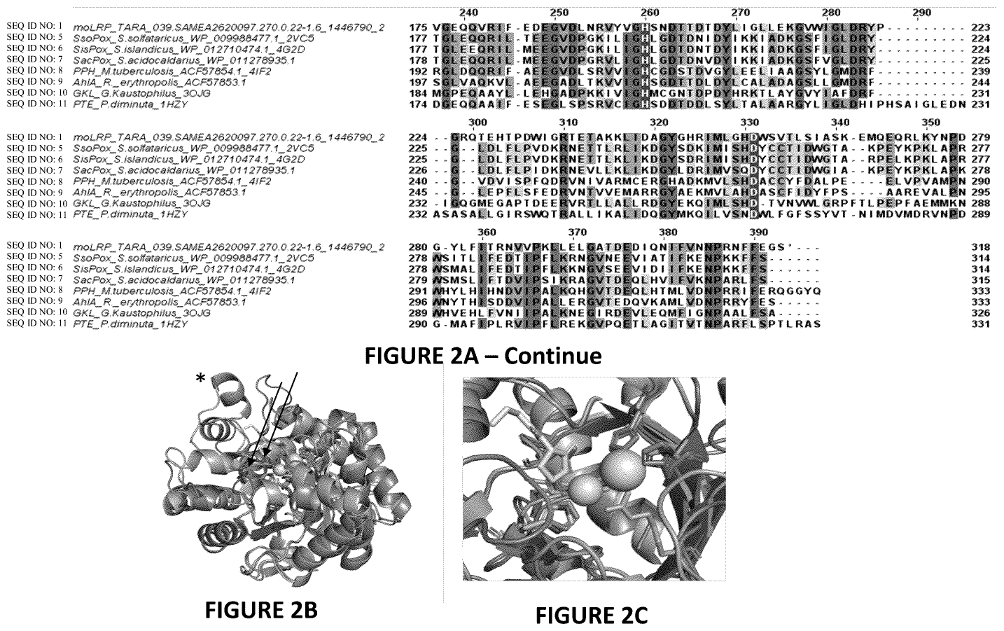

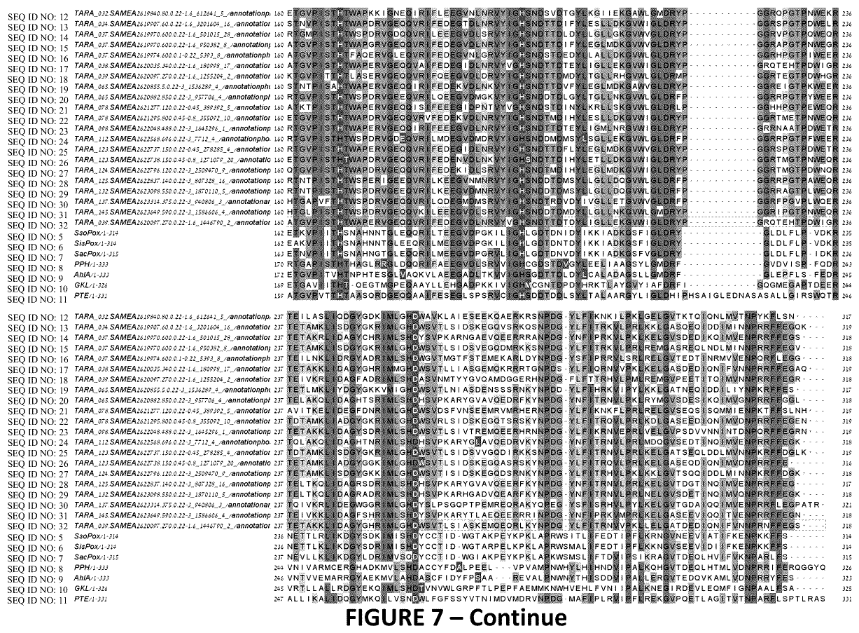

New putative AHL lactonases homologous genes were identified based on sequence homology to SsoPox from S. solfataricus within the 244 metagenomes from the Tara Oceans project. From these sequences, 19 sequences sharing 30-40% identity with SsoPox were selected based on the presence of conserved metal ions ligating residues in the PLL family, see alignment in FIG. 7. A phylogenetic tree of newly identified putative PLLs and previously characterized PLLs indicates the putative marine PLLs are grouped to a different clade than the previously characterized PLLs from a terrestrial origin (FIG. 1). The amino acid sequence alignment of one of the newly identified enzymes, a marine putative lactonase dubbed here moLRP (marine origin Lactonase Related Protein), with previously characterized terrestrial PLLs such as PPH, AhlA, SsoPox(66, 67) and GKL is presented in FIG. 2A. The structural homology model of moLRP was created and structural alignment with a thermostable quorum-quenching lactonase from Geobacillus kaustophilus GKL (pdb 3ojg), sharing 35% sequence identity, indicated both enzymes maintain the same fold (FIG. 2B). Five of the zinc-ligating residues His23, His25, His178, and His266, and Asp301 aligned perfectly, as well as the conserved sixth ligating residue, the carbamylated Lys 145 (numbering are according to GKL), see FIG. 2C.

The inventors examined the abundance of moLRP by aligning it against predicted proteins from in-house assemblies of the Tara samples, and against NCBI's nr database. Three nearly identical proteins (>99% identity) to moLRP were discovered in samples collected from three Tara stations in the Arabian sea. The samples were collected from depth of 270-600 meter below sea level. Two >99% hits were identified in NCBI's nr database, both from the Tara samples. One of these hits, MQG57092.1, is part of the Metagenomics Assembled Genome (MAG) GCA_009392485.1_ASM939248v1. The MAG is estimated to be 95% complete with 2% contamination (using checkm). Its NCBI taxonomic assignment is inconclusive, however assigning taxonomy using gtdbtk revealed that the MAG belongs to the phylum Chloroflexi (GTDB phylum Chloroflexota).

### Example 2

The New moLRP is an Efficient AHL Lactonase with a Preference Towards Short to Medium AHL-Chain

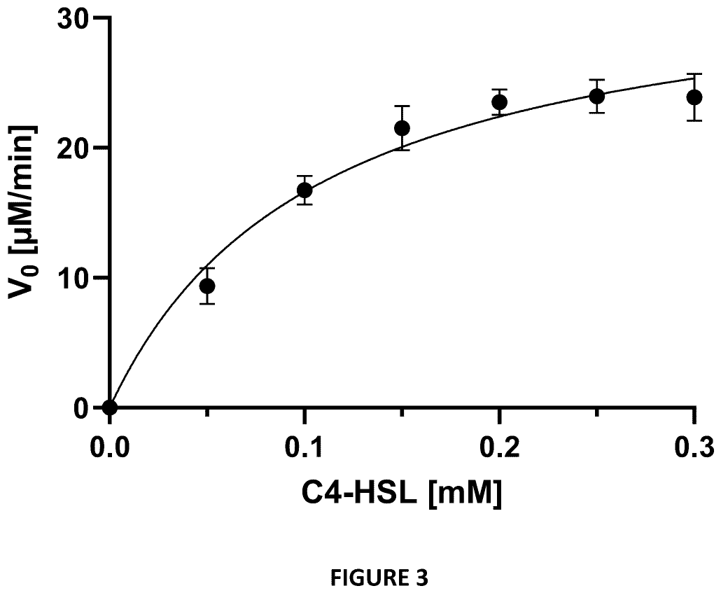

The synthetic gene of two putative enzymes were ordered cloned into pET-21 for expression in E. coli. Only one of them, moLRP, successfully over-expressed in E. coli culture with media containing different divalent metal ions, (Mn²⁺, Zn²⁺, and Co²⁺) that prevail in the amidohydrolase superfamily. The highest lactonase activity in lysates, tested with TBBL was observed with ZnCl₂ (data not shown). Therefore, for detailed characterization, moLRP was expressed in a large scale with supplemented ZnCl₂, and the protein was purified as described in the Methods section, using the 6×His tag. Purified protein yields were about 8 mg per 1 liter of E. coli culture, and the apparent molecular mass of the unique band observed on SDS-PAGE was 35 kDa. Its AHL lactonase activity was tested with a variety of AHLs. moLRP presented high activity with medium chain AHLs such as 3-oxo C6-HSL, C7-HSL and 3-Oxo C8-HSL, with k_{cat}/K_{M} values of 8.64×10³ s⁻¹·M⁻¹, 2.12×10³ s⁻¹·M⁻¹, 6.94×10³ s⁻¹·M⁻¹, respectively. The highest specific activity was detected with the short chain AHL; C4-HSL with k_{cat}/K_{M} value of 1.92×10⁴ s⁻¹·M⁻¹, see Table 1 and FIG. 3. These activities were compared to the previously tested activities of PPH12.

TABLE 1
The kinetic parameters for hydrolysis of variety AHLs by moLRP^{a}

| | | | k_{cat}/K_{M} |
|---|---|---|---|
| | K_{M}[μM] | k_{cat} [s⁻¹] | [M⁻¹ · S⁻¹] |
| C4-HSL | 96.54 ± 15.30 | 1.85 ± 0.11 | 1.92 × 10⁴ |
| 3-Oxo C6-HSL | 150.0 ± 13.00 | 1.30 ± 0.10 | 8.64 × 10³ |
| C7-HSL | 112.5 ± 19.00 | 0.230 ± 0.02 | 2.12 × 10³ |
| 3-Oxo C8-HSL | 74.77 ± 10.99 | 0.519 ± 0.02 | 6.94 × 10³ |
| C10-HSL | ND | ND | ND |

^{a}ND; no activity detected

### Example 3

moLRP Exhibits Stability and Tolerance in a Wide Range of Temperatures, pH, and Salinity

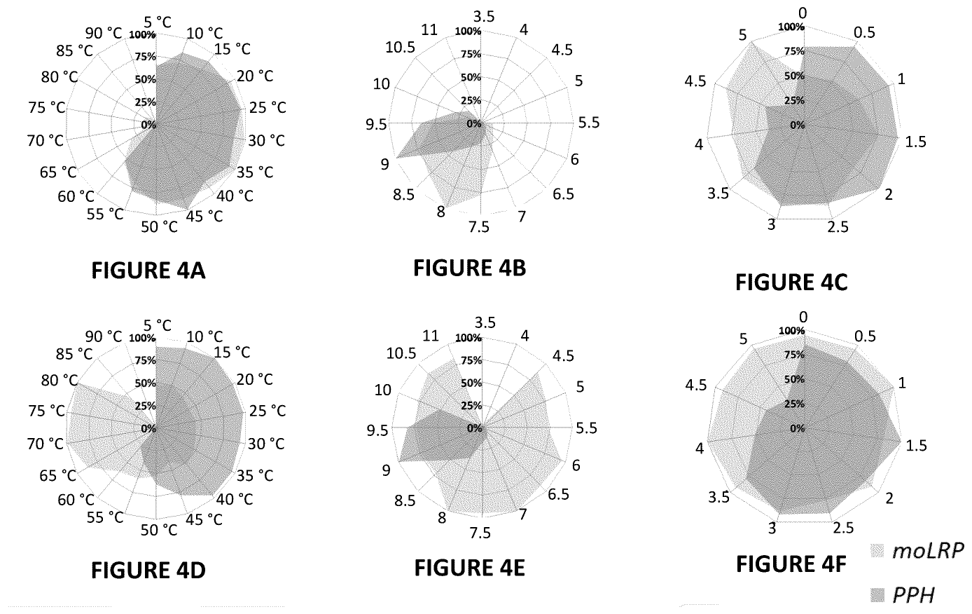

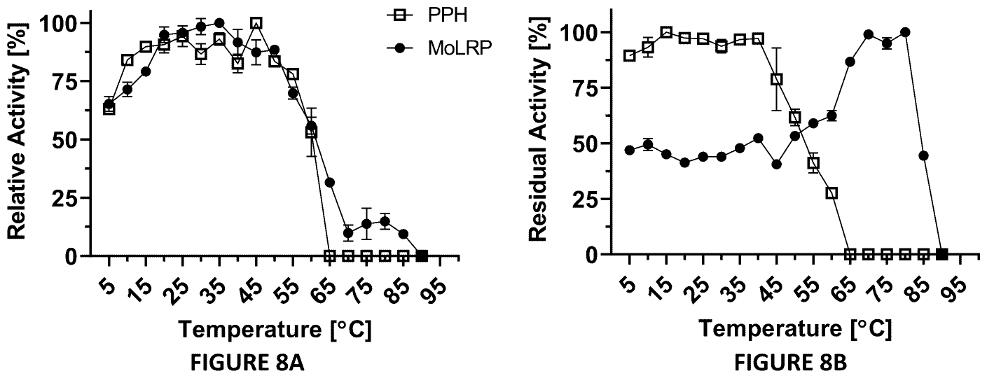

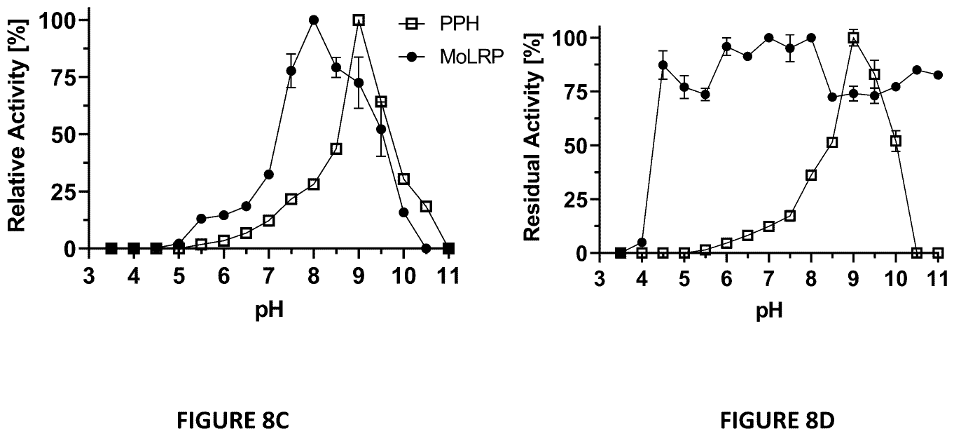

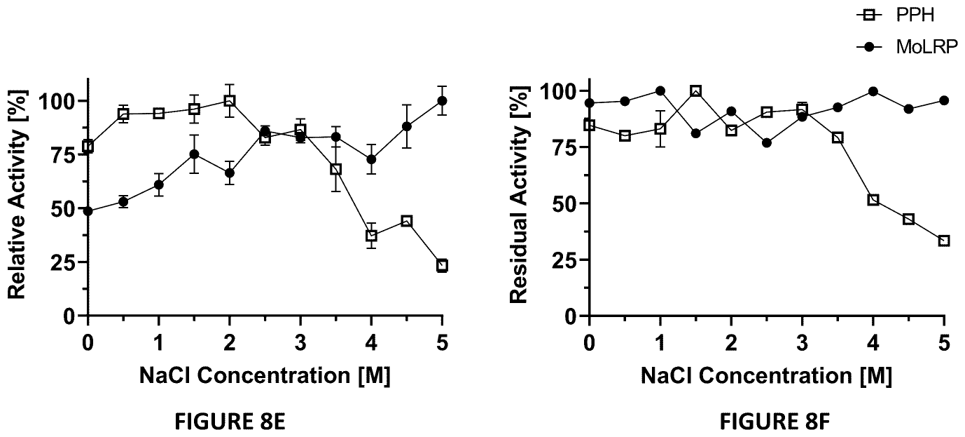

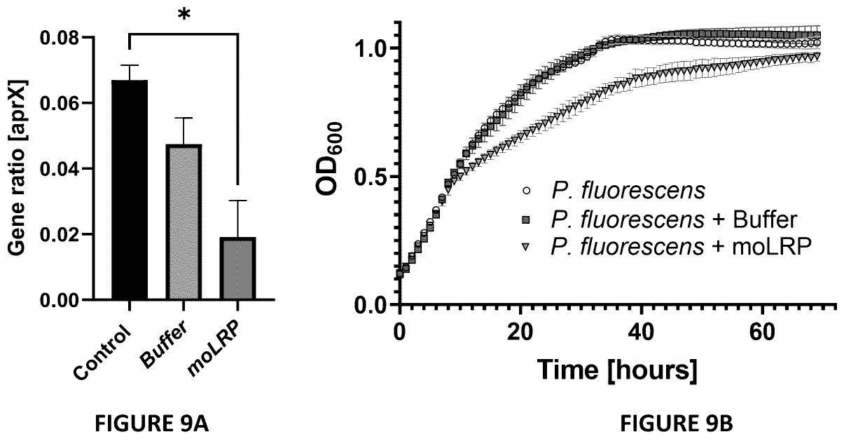

The biochemical characterizations indicate that moLRP differs in all most all parameters from the terrestrial PPH, especially in its stability at higher temperatures, and higher stability in a wide range of pH and salinity levels, see FIG. 4. Specifically, both moLRP and PPH exhibit 75% of its relative activity at a wide range of temperatures 10 to 55° C., as can be seen in FIG. 4A and FIG. 8A (presenting average values and SE from 3 repeats). moLRP also presented more than 75% activity between pH level of 7.5 to 8.5, with highest activity at pH 8, while PPH showed high activity at pH 9, FIG. 4B and FIG. 8B. More interestingly, the moLRP activity increased at high salinity levels, with highest activity detected at 5 M NaCl, while the relative activity of PPH at this salinity level was less than 25%. The marine enzyme exhibited resilience following incubation at temperatures above 65° C., but not in lower temperatures, FIG. 4D and FIG. 8D. PPH on the other hand, maintained more than 75% of its residual activity at low temperatures up to 45° C. and then declined drastically. While PPH maintained its activity following incubation of a narrow pH range around 9, moLRP presented more than 75% residual activity following an incubation at an extremely wide range of pH both acidic and basic (4.5-11), FIG. 4E and FIG. 8E. Furthermore, moLRP maintained close to 100% residual activity after incubation at all levels of salinity, 0 to 5 M NaCl (NaCl solubility limit), see FIG. 4F and FIG. 8F.

### Example 4

moLRP Inhibits Extracellular Proteolysis, Biofilm Formation, and Precipitation in Milk-Based Cultures of Pseudomonas fluorescens

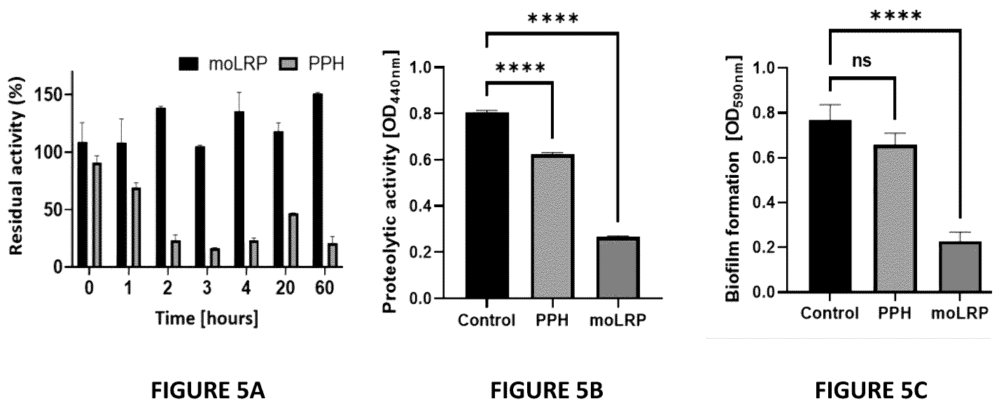

The inventors have grown cultures of P. fluorescens overnight in the presence of exogenically purified enzymes and bacterial cultures alone as control, in medium with skim milk. To have an indication that the enzymes are still active upon incubation in bacterial cultures, the inventors tested the residual lactonase activity of both PPH and moLRP with TBBL following their incubation in bacterial cultures, compared to their activity in buffer. As shown in FIG. 5A, moLRP maintain 100% of its activity for 60 hours while PPH lost about 80% of its activity after only 2 hours. Extracellular proteolytic activity of P. fluorescens cultures was tested with and without the addition of purified lactonases, FIG. 5B shows the proteolytic activity in supernatants of P. fluorescens. While the addition of purified PPH inhibited the bacterial proteolytic activity in skim milk by about 20%, moLRP inhibited 75% of the proteolytic activity, compared to supernatants from bacterial cultures without the addition of lactonase. Moreover, FIG. 5C shows that compared to bacterial cultures without the addition of lactonase, biofilm formation was inhibited 60% when purified moLRP was added to the cultures of P. fluorescens, and less than 20% when PPH was added.

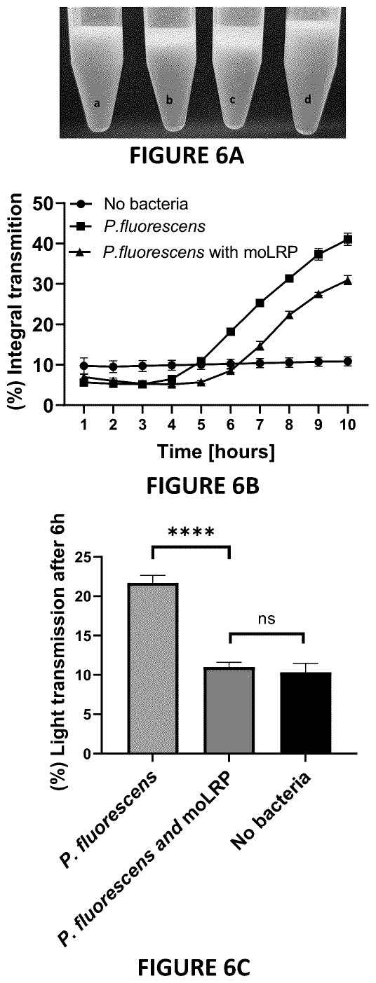

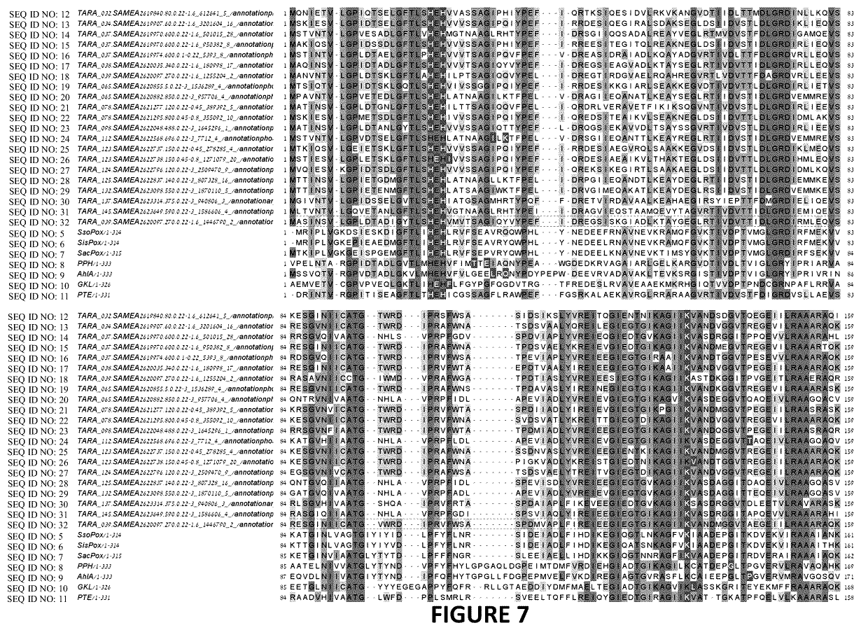

The effect on particles precipitation in milk cultures caused by P. fluorescens after 4 days, is presented in FIG. 6A. The presence of the exogenically added purified PPH to bacterial cultures had no significant effect on the visible precipitation process. However, cultures incubated with moLRP appeared homogeneous as skim-milk medium without bacteria. To better evaluate the degree of particles precipitation, similar to the process that occurs during bacterial born milk spoilage, sedimentation progress of skim milk-based cultures of P. fluorescens was also evaluated by clarification of the upper part of 10% skim-milk based cultures, with and without purified moLRP. In FIG. 6B it is shown that while control samples containing skim-milk medium alone remained homogenous even after 10 h, skim-milk medium inoculated with P. fluorescens undergone destabilization and sedimentation processes after 4 hours. However, the addition of purified moLRP to skim-milk medium inoculated with P. fluorescens cultures resulted in a 2 h delay of in sedimentation (from 4 h to 6 h). FIG. 6C presents the average light transmission after 6 hours, showing that at that time, the skim-milk alone control and P. fluorescens culture with moLRP showed similar values, whereas the light transmission of samples with P. fluorescens culture alone was significantly larger (p<0.0001), indicating that the destabilization and sedimentation process of the milk medium is inhibited by moLRP.

The inventors further showed that the activity of the moLRP enzyme disclosed herein specifically results in reduced relative expression levels (e.g., transcription) of QS-regulated genes, such as aprX (encoding a proteinase; 9A) and does not substantially affect bacterial growth (9B).

The invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. An isolated polypeptide comprising the amino acid sequence: HEHVMVTSAGIQYVYPEFIDREGSISKGIADLKTAYGEGLRTIVDVTTIDLGRDIR MLEQVSRESGINIICATGVWRDIPRVFWSASPDMVAPLFIREIEEGIEGTGIKAGIIK VANDMGGVTAEGEIILRAAARAQKATGVPISTHTWAPERVGEQQVRIFEDEGVD LNRVYVGHSNDTTDTDYLIGLLEKGVWIGLDR YPGRQTEHTPDWIGRTETAKKLI DAGYGHRIMLGHDWSVTLSIASKEMQEQRLKYNPDGYLFITRNVVPKLLELGAT DED (SEQ ID NO: 3), or a functional analog having at least 95% homology or identity thereto.

2. The isolated polypeptide of claim 1, comprising the amino acid sequence set forth in SEQ ID NO: 1, or a functional analog having at least 95% homology or identity thereof, and optionally wherein said isolated polypeptide is a quorum-quenching N-acyl-homoserine lactonase.

3. The isolated polypeptide of claim 1, characterized by any one of: (i) having an activity at pH ranging from 7.5 to 9.5; (ii) having an activity at a salinity ranging from 0 to 5 M; (iii) having an activity at a temperature ranging from 1 to 60° C.; (iv) being stable at temperature ranging from 1 to 85° C.; (v) being stable at a pH ranging from 4.5 to 11.0; (vi) being stable at a salinity ranging from 0 to 5 M; and (vii) any combination of (i) to (vi).

4.-5. (canceled)

6. The isolated polypeptide of claim 3, wherein said activity comprises any one of: (i) at least 50% activity compared to an optimal activity control; (ii) ester bond hydrolysis, optionally wherein said ester bond is an ester bond of a homoserine lactone ring, and optionally wherein said homoserine lactone is an acylated homoserine lactone; and both (i) and (ii).

7.-9. (canceled)

10. The isolated polypeptide of claim 6, wherein said stable is comprising or maintaining at least 50% of the activity compared to optimal conditions.

11.-14. (canceled)

15. A polynucleotide encoding the isolated polypeptide of claim 1.

16. The polynucleotide of claim 15, comprising a nucleic acid sequence being codon optimized for expression in a cell, optionally wherein said nucleic acid sequence being codon optimized for expression in a cell comprises the nucleic acid sequence set forth in SEQ ID NO: 2, and optionally wherein said cell is a bacterial cell or a fungal cell.

17.-18. (canceled)

19. An artificial nucleic acid molecule or vector comprising the polynucleotide of claim 15, and optionally wherein said artificial nucleic acid molecule or vector being a plasmid or an expression vector.

20. (canceled)

21. A cell comprising the isolated polypeptide of claim 1, and optionally wherein any one of: (i) said cell being any one of: a unicellular organism, a cell of a multicellular organism, and a cell in a culture; (ii) said cell being a transgenic cell, a transformed cell, or a transfected cell; and both (i) and (ii).

22.-23. (canceled)

24. An extract obtained or derived from the cell of claim 21, and optionally wherein said extract comprises an isolated polypeptide comprising the amino acid sequence set forth in SEQ ID NO: 3, or a functional analog having at least 95% homology or identity thereto.

25. (canceled)

26. A composition comprising the isolated polypeptide of claim 1, and an acceptable carrier, optionally wherein said composition being characterized by being capable preventing or reducing food spoilage, and optionally wherein any one of: said food spoilage is induced by or involves any one of a bacterium and biofilm produced by same, and said bacterium comprises Pseudomonas fluorescens.

27.-29. (canceled)

30. An article comprising the isolated polypeptide of claim 1, optionally wherein any one of: (i) said isolated polypeptide is bound to a surface of said article, and optionally wherein said bound is covalently bound; (ii) said article being configured to storing or transferring a liquid; and both (i) and (ii).

31.-33. (canceled)

34. A method of inhibiting or reducing a formation of load of organic-based contaminant on or within an article, comprising coating or incorporating a composition comprising the isolated polypeptide of claim 1 to said article, thereby inhibiting or reducing a formation of load of organic-based contaminant on or within the article, and optionally wherein any one of: (i) said incorporating comprises binding said isolated polypeptide to a surface of said article, and optionally wherein said binding comprises covalently binding; (ii) said article is configured to storing or transferring a liquid, and optionally wherein said liquid comprises water; (iii) said article comprises any one of: a tubing, a capsule, and a container; and (iv) any combination of (i) to (iii).

35.-39. (canceled)

40. A method of inhibiting or reducing a formation of load of organic-based contaminant in a composition, the method comprising contacting said composition with an effective amount of the isolated polypeptide of claim 1.

41. The method of claim 40, wherein any one of: (i) said composition is susceptible to formation of said organic based contaminant; (ii) said composition is an edible composition, optionally wherein said edible composition is a food product, and optionally wherein said food product comprises any one of a dairy product and a meat product; and (iii) both (i) and (ii).

42.-44. (canceled)

45. The method of claim 34, wherein said organic-based contaminant comprises biofilm.

46. The method of claim 34, wherein said organic-based contaminant comprises the bacterium P. fluorescens, and optionally wherein said biofilm is produced by P. fluorescens.

47. (canceled)

48. The method of claim 40, wherein said organic-based contaminant comprises biofilm.


SEQUENCE LISTING (32 sequences)

SEQ ID NO: 1 - protein, 317 aa, synthetic construct
  REGION 1..317: note = Synthetic
MASINSVLGPLDTADIGYTLSHEHVMVTSAGIQYVYPEFIDREGSISKGIADLKTAYGEGLRTIVDVTTIDLGRDIRMLEQVSRESGINIICATGVWRDIPRVFWSASPDMVAPLFIREIEEGIEGTGIKAGIIKVANDMGGVTAEGEIILRAAARAQKATGVPISTHTWAPERVGEQQVRIFEDEGVDLNRVYVGHSNDTTDTDYLIGLLEKGVWIGLDRYPGRQTEHTPDWIGRTETAKKLIDAGYGHRIMLGHDWSVTLSIASKEMQEQRLKYNPDGYLFITRNVVPKLLELGATDEDIQNIFVNNPRNFFEGS

SEQ ID NO: 2 - DNA, 951 bp, synthetic construct
  misc_feature 1..951: note = Synthetic
  source 1..951: mol_type = other DNA
atggcgaccattaacagcgttctgggtccgctggacaccgcggacatcggctacaccctgagccacgagcacgtgctggttaccagcgcgggtatccagtacgtgtatccggagttcatcgaccgtgaaggcaccattgcgcgtgcggttgcggatctgaagaccgcgtatagcgaaggtctgcgtaccatcgtggacgttaccaccattgacctgggccgtgacatccgtatgctggagcaagtgagccgtgaaagcggtattaacatcatttgcgcgaccggcacctggcgtgacatcccgcgtgtgttttggagcgcgagcccggatatggttgcgccgctgtacatccgtgagattgaggaaggtatcgaaggcaccggcattaaggcggcgatcattaaagttgcgaacgatgtgggtggcgttaccccggagggcgaaatcattctgcgtgcggcggcgcgtgcgcagaaggcgaccggtgtgccgattagcacccatacctgggcgccggagcgtgtgggtgaacagcaagttcgtatcttcgaggacgaaggtgttgatctgaaccgtgtgtacgttggccacagcaacgacaccaccgataccgagtatctgattggtctgctggaaaaaggtgtgtggatcggcctggaccgttatccgggtcgtcagaccgagcacaccccggattggatcggccgtaccgaaaccgcgaagaaactgattgacgcgggttatggccaccgtatcatgctgggtcacgattggagcgttaccctgagcattgcgagcaaggagatgcaggaacaacgtctgaaatacaacccggacggttacctgttcatcacccgtaacgtggttccgaagctgaaagagctgggcgcgaccgaggaagacatccagaacatttttgttaacaacccgcgtaacttctttgaagcgagc

SEQ ID NO: 3 - protein, 280 aa, synthetic construct
  REGION 1..280: note = Synthetic
HEHVMVTSAGIQYVYPEFIDREGSISKGIADLKTAYGEGLRTIVDVTTIDLGRDIRMLEQVSRESGINIICATGVWRDIPRVFWSASPDMVAPLFIREIEEGIEGTGIKAGIIKVANDMGGVTAEGEIILRAAARAQKATGVPISTHTWAPERVGEQQVRIFEDEGVDLNRVYVGHSNDTTDTDYLIGLLEKGVWIGLDRYPGRQTEHTPDWIGRTETAKKLIDAGYGHRIMLGHDWSVTLSIASKEMQEQRLKYNPDGYLFITRNVVPKLLELGATDED

SEQ ID NO: 4 - DNA, 840 bp, synthetic construct
  misc_feature 1..840: note = Synthetic
  source 1..840: mol_type = other DNA
cacgagcacgtgctggttaccagcgcgggtatccagtacgtgtatccggagttcatcgaccgtgaaggcaccattgcgcgtgcggttgcggatctgaagaccgcgtatagcgaaggtctgcgtaccatcgtggacgttaccaccattgacctgggccgtgacatccgtatgctggagcaagtgagccgtgaaagcggtattaacatcatttgcgcgaccggcacctggcgtgacatcccgcgtgtgttttggagcgcgagcccggatatggttgcgccgctgtacatccgtgagattgaggaaggtatcgaaggcaccggcattaaggcggcgatcattaaagttgcgaacgatgtgggtggcgttaccccggagggcgaaatcattctgcgtgcggcggcgcgtgcgcagaaggcgaccggtgtgccgattagcacccatacctgggcgccggagcgtgtgggtgaacagcaagttcgtatcttcgaggacgaaggtgttgatctgaaccgtgtgtacgttggccacagcaacgacaccaccgataccgagtatctgattggtctgctggaaaaaggtgtgtggatcggcctggaccgttatccgggtcgtcagaccgagcacaccccggattggatcggccgtaccgaaaccgcgaagaaactgattgacgcgggttatggccaccgtatcatgctgggtcacgattggagcgttaccctgagcattgcgagcaaggagatgcaggaacaacgtctgaaatacaacccggacggttacctgttcatcacccgtaacgtggttccgaagctgaaagagctgggcgcgaccgaggaagac

SEQ ID NO: 5 - protein, 299 aa, Sulfolobus solfataricus
MRIPLVKDSIESKDIGFTLIHEHRVFSWPHLYNEDEEFRNAVNEVKRAMQFGVKTIVDPTVMGLGRDIRFMEKVVKATGINLVAGTGIYIDLPFYFLNRSIDEIADLFIHDIKEGIQGTLNKAGFVKIAADPGITKDVEKVIRAAAIANKETKVPIITHSNAHNTGLEQQRILTEEGVDPGKILIGHLGDTDNIDYIKKIADKGSFIGLDRYGLDLFLPVDKRNETTLRLIKDGYSDKIMISHDYCCTIDWGTPEYKPKLAPRSITLIFEDTIPFLKRNGVNEEVIATIFKENPKKFFS

SEQ ID NO: 6 - protein, 299 aa, Sulfolobus islandicus
MRIPLVKEPIEAEDMGFTLIHEHRVFSWPHLYNEDEELRNAVNEVKRAMQFGVKTIVDPTVMGLGRDIRFMEKVVKTTGINLVAGTGIYIDLPFYFLNRSIDEIADLFIHDIKEGIQATSNKAGFVKIAADPGITKDVEKVIRAAAITHKEAKVPIITHSNAHNTGLEEQRILMEEGVDPGKILIGHLGDTDNTDYIKKIADKGSFIGLDRYGLDLFLPVDKRNETTLKLIKDGYSDRIMISHDYCCTIDWGTPELKPKLAPRSMALIFEDTIPFLKKNGVSEEVIDIIFKENPKKFFS

SEQ ID NO: 7 - protein, 299 aa, Sulfolobus acidocaldarius
TKIPLVKGEISPGEMGFTLIHEHRVFSWPHLYNEDEELKNAVNEVKTIMSYGVKTIVDPTVMGLGRDIRFSEKVVKETGINVIAATGLYTDLPFFFNGRSLEEIAELLIHDIKKGIQGTNNRAGFIKVAADPGITRDVERAIRAAAIAQKETNVPIITHSNAHNTGLEQQRILMEEGVDPGRVLIGHLGDTDNVDYIKKIADKGSFVGLDRYGLDLFLPIDKRNEVLLKLIKDGYLDRIMVSQDYCCTIDWGIPEYKPKLAPKSMSLIFTDVIPSIKRAGVTDEQLHVIFVKNPARLFS

SEQ ID NO: 8 - protein, 299 aa, Mycobacterium tuberculosis
PELNTARGPIDTADLGVTLMHEHFIMTYPEAWDEDKRVAGAIARLGELKARGVDTIVDLTVIGLGRYIPRIARVAAATELNIVVATGLYTDVPFYFHYLGPEIMTDMFVRDIEHGIADTGIKAGILKCATDPGLTPGVERVLRAVAQAHKRTGAPISTHTHAGLRGLDQQRIFAEEGVDLSRVVIGHCGDSTDVGYLEELIAAGSYLGMDRFGVDVISPFQDRVNIVARMCERGHADKMVLSHDACCYFDALPEELVPVAMPNHYLHIHNDVIPALKQHGVTDEQLHTMLVDNPRRIFE

SEQ ID NO: 9 - protein, 299 aa, Rhodococcus erythropolis
SSVQTVRGPVDTADLGKVLMHEHFVLGYPDYWDEEVRVADAVAKLTEVKSRGISTIVDPTVIGLGRYIPRIVRINEQVDLNIIVATGIYTDIPFQFHYTGPEPMVELFVKDIREGIAGTGVRASFLKCAIEPGLTPGVERVMRAVGQSQVETGVPITVHTNPHTSGLVAQKVLAEEGADLTKVVIGHSGDTTDLDYLCALADAGSLLGMDRFGLEPFLSFEDRVNTVVEMARRGYAEKMVLAHDASCFIDYFPSAAREVALPNNYTHISDDVIPALLERGVTEDQVKAMLVDNPRRYFE

SEQ ID NO: 10 - protein, 299 aa, Geobacillus kaustophilus
EMVETVCGPVPVEQLGKTLIHEHFLFGYPGFQDEDESLRVAVEAAEKMKRHGIQTVVDPTPNDCGRNPAFLRRVAEETGLNIICATGYYYEAPPYFQFRRLDDIYDMFMAELTEGIADTGIKAGVIKLASSGRITEYEKMFFRAAARAQKETGAVIITHTQEGTMGPEQAAYLLEHGADPKKIVIGHMCGNTDPDYHRKTLAYGVYIAFDRFGMEGAPTDEERVRTLLALLRDGYEKQIMLSHDTVNVWLGRPPEPFAEMMKNHVEHLFVNIIPALKNEGIRDEVLEQMFIGNPAALFS

SEQ ID NO: 11 - protein, 299 aa, Pseudomonas deminuta
DRINTVRGPITISEAGFTLTHEHCGSSWPEFFSRKALAEKAVRGLRRARAAGVRTIVDVSTFDIGRDVSLLAEVSRAADVHIVAATGLWFDPPLSMRLRSVEELTQFFLREIQYGIEDTGIRAGIIKVATTGKATPFQELVLKAAARASLATGVPVTTHTAASQDGEQQAAIFESEGLSPSRVCIGHSDDTDDLSYLTALAARGYLIGLDHIGLEDNASWQTRALLIKALIDQGYMKQILVSNDWLFGFSSYVMDVMDRVNPDGMAFIPLRVIPFLREKGVPQETLAGITVTNPARFLS

SEQ ID NO: 12 - protein, 305 aa, unidentified
  source 1..305: note = Bacteria
QNIETVLGPIQTSELGFTLSHEHVVASSAGIPHIYPEFIQRTKSIQESIDVLRKVKANGVDTIIDLTTMDLGRDINLLKQVSKESGINIICATGTWRDIPRSFWSASIDSIKSLYVREITQGIENTNIKAGIIKVANSDGVTQEGEIILRAAARAQIETGVPISTHTWAPKIGNEQIRIFLEEGVNLNRVYIGHSNDSVDTGYLKGIIEKGAWLGMDRYGQPGTPNWEKRTEILASLIQDGYGDKIMLGHDAVKLAIESEAERKRSNPDGYLFIKNKILPKLGELGVTKTQIQNLMVTNPYKFLS

SEQ ID NO: 13 - protein, 305 aa, unidentified
  source 1..305: note = Bacteria
SKIESVLGPIDTSELGFTLSHEHVVVSSAGIPQIYPEFIRRDESIQEGIRALSDAKSEGLDSIIDVSTLDLGRDIKLIEQVSRESGVNIICATGTWRDIPRAFWNATSDSVAALYQREILEGIEGTGIKAGIIKVANVGGVTREGEVILRAAARAQKSTNVPISTHTWAPDVGEQQVRIFEDEGTDLNRVYIGHSNDTTDIFYLESLLDKGVWIGLDRYGSPGTPDWELRTETAMKLISDGYGKRIMLGHDSVTLSIDSKVSRKQSNPDGYLFITRKVLPRLKKLGASQEQIDDIMINNPRRFFE

SEQ ID NO: 14 - protein, 305 aa, unidentified
  source 1..305: note = Bacteria
STVNTVLGPVESADLGFTLVHEHMGTNAAGLRHTYPEFIDRSGIIQQSADALREAYVEGLRTMVDVSTFDLGRDIGAMQEVSRRSGVQIVAATGNHLSVPRPFGDVSPDVIAPLYVREIEQGIEDTGVKAGIIKVASRGGITPPQEVVLRAAARAHLRTGMPISTHTWSPDVGDQQVRILEEEGVDLSRVYIGHSNDTTDLDYLLGLLGKGVWVGLDRYGVPGTPQWEQRTETAKRLIDAGYCHRIMLSHDSVPKARNGAEERRRANPDGYNFIPRKVLPKLKELGVSDEDIKQIMVENPRRFFE

SEQ ID NO: 15 - protein, 305 aa, unidentified
  source 1..305: note = Bacteria
AKIQSVLGPIDSSDLGFTLSHEHVVVSSAGIPHIYPEFIRREESITEGITQLREAKNEGLDSIIDVSTIDLGRDIRLIEQVSRESGINIICATGTWRDIPRAFWNATPDSVAELYKREIEVGIEDTGIKAGIIKVANMEGVTREGEVILRAAARTQKATGVPISTHTWAPGVGEQQVRIFEDEGVDLNRVYIGHSNDTTDIFYLEGLMDKGVWVGLDRYGAPGTPNWEGRTETAMKLISDGYGKRIMLGHDSVTLSIDSVDMRKKSNPDGYLFITRKVLPRLREMGASREQLNDLMINNPRRFFE

SEQ ID NO: 16 - protein, 305 aa, unidentified
  source 1..305: note = Bacteria
STINTVLGPLDTAELGFTLPHEHVVVTSAGIPQVFPEFVDREASIDRAIADLKQAYADGVRTIVDLTTFDLGRDVRLLEQVSRDSGVQIICATGTWRDIPRVFWSATPEVIAPLYVREIEEGIEGTGIRAAIIKVANTGGVTPESEVVLRAAARAQKATGVPISTHTFAQEVGLEQVRIFEDEGVNLGRVYIGHSNDSTDEDYLLALLDKGVWLGLDRYGHPGTPIWEERTEILNKLIDRGYADRIMLSHDGVTMGTFSTKARLDYNPDGYSFIPRRVLSRLRELGVTEDTINRMVVENPQRFLE

SEQ ID NO: 17 - protein, 305 aa, unidentified
  source 1..305: note = Bacteria
AQINSVLGPIDTADLGFTLSHEHVVVTSAGIQYVYPEFIDREGSIEAGVADLKTAYSEGLRTIVDVTTIDLGRDIRLLEQVSRESGINIICATGTWRDIPRVFWTAEPDTVAALYIREIEEGIEGTGIKAAIIKVANVGGVTPEGEIILRAAARAQKATGVPISTHTWAPEVGEQQVAIFEDEGIDLNRVYVGHSNDTTDTEYLIGLLEKGVWIGLDRYGTEHTPDWIGRTETAKKLIDAGYGHRIMMGHDSVTLLIASGKRRRTGNPDSYLFIQRNVVPKLKELGASDEDIQNIFVNNPRRFFE

SEQ ID NO: 18 - protein, 305 aa, unidentified
  source 1..305: note = Bacteria
ANVNTVLGPLDTSKLGFTLAHEHILPTSAGIQQVYPELIDREGTIRDGVAELRQAHREGVRTLVDVTTFDAGRDVRLLEEVSRASAVNIICCTGIWMDVPRAFWGATPDTIASLYVREIEESIEGTGIKAGIIKASTKGGITPVGEITLRAAERAQRKTGVPITTHTLASEVGEQQVRIFQEEGVDLDRVYIGHSNDTTDMDYLTGLLRHGVWLGLDRMGTEGTPDWHGRTEIVKRLIDAGFADRIMLSHDSVNMTYYGVDGGERHNPDGFLFTTRHVLPMLREMGVPEETITQIMVNNPRRFFE

SEQ ID NO: 19 - protein, 305 aa, unidentified
  source 1..305: note = Bacteria
TSIQTVLGPIDTSKLGQTLSHEHVIVSSGGIPQIYPEFIRRDESIKKGIARLSEAKKEGVDTIVDVTTLDLGRDIRLIETVSRESGVNIICATGTWRDIPRAFWNSTPDAVASLYIREINEGIEGTGIKAGIIKVANKGGVTREGEVILRAAARASKSTNTPISAHTWAPEVGEQQIRIFEDEKVDLNKVYIGHSNDTTDVFYLEGLMDKGAWVGLDRYGEIGTPKWEERTETLMKLIYDGYGKKVMIGHDSVTLNIASDSSRNKYNPDDYLFIHRKVIPYLKKLGATNEQIDDILIYNPKRFFE

SEQ ID NO: 20 - protein, 305 aa, unidentified
  source 1..305: note = Bacteria
PAVNTVLGPVETEDLGFTLSHEHLATNAAGILKTFPELVDRAGIIEQANATLKEAYSEGLRTIIDVSTIDLGRDVEMMREVSQNTRVNIVAATGNHLAVPRPFIDLAPEVIADLYVREIEVGIEGTGIKAGVIKVASEGGVTQAQEIVLRAAGQACVRTGVPISTHTWSPDVGEEQVRILQEEGVDMGKVYIGHSNDDTDLGYLTGLLNKGVWLGLDRFGRPGTPYWEERTELAKQLIDAGHTSRIMLSHDSVPKARYGVEDRFKYNPDGYNFITRNVLPKLRYMGVSDESIKQIMVGNPRRFFG

SEQ ID NO: 21 - protein, 305 aa, unidentified
  source 1..305: note = Bacteria
ATINSVLGPLDTGNLGFTLSHEHILTTSAGIQQVFPELIQRDRLVERAVETFIKIKSQGVNTIVDVSTLDLGRDIRLIEEVSKRSGVNVICATGTWRDIPRVFWTASPDIIADLYIREIEVGIEGTGIKPGIIKVANMGGVTPEGEIILRAASRASKATKTPISTHTWAPEVGEQQISIFEEEGIDPNTVYVGHSNDSTDINYLKRLLSKGVWLGLDRLGHPGTPEWEQRAVITKELIDEGFDNRIMLGHDSVDSFVNSEVMRHERNPDGYLFIKNKFLPRLRELGVSEQSISQMMIENPKTFFS

SEQ ID NO: 22 - protein, 305 aa, unidentified
  source 1..305: note = Bacteria
SKIESVLGPMETSDLGFTLSHEHIVVSSAGIPQIYPEFIQREDSITEAIKVLSEAKNEGLDSIIDVTTLDLGRDIDMLAKVSKESGVNIICATGTWRDIPRVFWNASVDSVATLYKREITTGIEGTDIKAGIIKVANVGGVTREGEVILRAAARAQKETGVPISTHTWAPDVGEQQVRVFEDEKVDLNRVYIGHSNDTMDIHYLESLLDKGVWIGLDRYGTMETPNWEQRTDTAMKLIADGYGKRIMLGHDSVRLSIDSKTSRSKYNPDGYLFITRQVLPRLKDLGASEEDIQNIMVDNPRRFFE

SEQ ID NO: 23 - protein, 305 aa, unidentified
  source 1..305: note = Bacteria
ATINSVLGPLDTANLGYTLSHEHVVVSSAGIQTTYPEFLDRQGSIEKAVVDLTSAYSSGVRTIVDVSTLDLGRDIRLIEEVSRRSGVNFIAATGTWRDIPRVFWTATPDMIADLYVREIEEGIEGTGIKAGIIKVANQGGVTPEGEIILRAASRAQRRTGVPISTHTWAPEVGEQQIRIFEEEGVDLNRVYIGHSNDTTDTEYLIGMLNRGVWIGLDRYGNAATPDWETRTETAKKLIDAGYSDRIMLSHDSVTLSIVTREERHRNNPDGYLFIKRNVEPRLVELGVPSDVVNNINTDNPQRFFE

SEQ ID NO: 24 - protein, 305 aa, unidentified
  source 1..305: note = Bacteria
STVNTVLGPVDTSELGFTLSHEHLATNAAGILKTFPELVDRSGIIEQANTTLKEAYREGLRTIIDVSTIDLGRDVEMMREVSKATGVHIIAATGNHLAVPRPFLDLAPEVIADLYVREIEVGIEGTGIKAGIIKVASEGGVTTAQEIVLRAAGQAQVRTGVPISTHTWSPDVGDEQVRILMDEGVDMDRVYIGHSNDDMDMSYLSGLLEKGVWLGLDRFGRPGTPFWEERTQLAKQLIDAGHTNRIMLSHDSVPKARYGLEDRVKYNPDGYNFITRNVLPRLMDQGVSEDTINQIMVENPRRFFE

SEQ ID NO: 25 - protein, 305 aa, unidentified
  source 1..305: note = Bacteria
TKIQSVLGEIETSKLGFTLSHEHVVVSSAGIPQIYPEFIRRDESIAEGVRQLSAAKKEGVDSIIDVSTLDLGRDIKLMEQVSKESGVNIICATGTWRDIPRAFWNASSDNVASLYVREIEEGIENTKIKAGIIKVANMGGVTREGEIILRAAARAQKRTGVPISTHTWAPEVGEQQVRIFEDEGIDLNRVYIGHSNDTTDFLYLEGLMDKGVWVGLDRYGSPGTPDWETRTDTAMKLISDGYGKRIMLGHDSVTLSIDSVDIRKKSNPDGYLFITRNVLPRLKELGATSEQLDDLMVNNPKRFFD

SEQ ID NO: 26 - protein, 305 aa, unidentified
  source 1..305: note = Bacteria
STIESVLGPLETSKLGFTLSHEHIVVSSAGIPQIYPEFIQRDESIAEAIKVLTHAKTEGLDSIIDVTTLDLGRDIHMLEQVSKESGVNIICATGTWRDIPRAFWNAPIGKVADLYKREISTGIEDTNIKAGIIKVANTGGVTREGEIILRAAARAQKETGVPISTHTWAPDVGEQQVRIFEDENVDLNKVYIGHSNDTTDIHYLETLLDKGVWIGLDRYGTMGTPNWEQRTETAMKLIADGYGGKIMLGHDSVTLSIDSKASRSKYNPDGYLFITRQVLPRLKELGASEQDIQNIMVDNPRRFFE

SEQ ID NO: 27 - protein, 305 aa, unidentified
  source 1..305: note = Bacteria
VQIESVKGPIDTSDLGFTLSHEHVVVSSAGIPQIYPEFIQREKSIKEGIRTLSAAKAEGLDSIIDVTTLDLGRDIDMLKQVSEGSGVNIVCATGTWRDIPRAFWSATSDSVAELYTREITVGIEGTDIKAGIIKVANVGGVTREGEIILRAAARAQKQTGVPISTHTWAPDVGEQQVRIFEDEKIDLSRVYIGHSNDTMDIHYLEGLMDKGVWIGLDRYGTMGTPDWNQRTETAMKLISDGYGKKIMLGHDSVTISIDSKDSRNKYNPDGYLFITRHVLPKLKKLGVTSQQIDDLMINNPKRFFD

SEQ ID NO: 28 - protein, 305 aa, unidentified
  source 1..305: note = Bacteria
TTINSVLGPIETGDMGFTLSHEHLATNAAGLLKTFPELIDRPGIIEQANDTLKEAYDEGLRTIIDVSTIDLGRDIEMMKEVSQNTGVQIIAATGNHLAVPRPFIDLSPEVISDLYVREIEEGIEGTGVKAGIIKVASAGGITDAQEIVLRAAGQASVRTNTPISTHTWSPEIGEEQVRILKEEGVNMNRVYIGHSNDDTDMSYLLGLLDQGVWLGLDRFGRPGTPAWEQRTELTKQLIDAGRSDRIMLSHDSVPKARYGVEERFKYNPDGYNFITRNVLPRLKELGVTDGTINQIMVENPRRFFE

SEQ ID NO: 29 - protein, 305 aa, unidentified
  source 1..305: note = Bacteria
TTINSVLGPIETENMGFTLSHEHLATSAAGIWKTFPELIDRVGIMEQAKATLKEAYDEGLRSIIDVSTIDLGRDVEMMKEVSGATGVQIVAATGNHLAVPRPFADLAPEVIADLYVREIEVGIEGTGVKAGIIKVASAGGITTEQEIVLRAAGQASVRTNTPISTHTWSPDVGEEQVRILKEEGVDMNRVYIGHSNDDTDMSYLLGLLDQGVWLGLDRFGRAGTPLWEQRTELTKQLIDAGRADRIMLSHDSVPKARHGAEARFKYNPDGYNFITRNVLPRLNELGVSDETINQIMVQNPRRFFE

SEQ ID NO: 30 - protein, 305 aa, unidentified
  source 1..305: note = Bacteria
GIVNTVLGPIDSSELGVTLSHEHIATGSAGMHRTYPQFFDRETVIRDAVAALKEAHAEGIRSYIEPTTFDMGRDIELIQEVSRLSGVHIVAATGSHQAIPRVFRTASPDAIAPLFIKEVEEGIEDTGVKAGIIKSASRGGVTDLEETVLRAVARASKHTGAPVFTHTWSPDVGEQQVRILQDEGMDMSRVYIGHSNDTRDVDYIVGLLETGVWVGLDRFGFPGVPLWQERTQIVKRLIDEGWGGRIMLGHDSLPSGQPTPEQRAKYQPEGYSFISRHVLPYLKELGVTGDAIQQIMVENPRRFLE

SEQ ID NO: 31 - protein, 305 aa, unidentified
  source 1..305: note = Bacteria
LTVNTVLGQVETANLGFTLSHEHVGTNAAGLRHTYPEIIDRAGVIEQSTAAMQEVYTAGVRTIVDVTTIDLGRDIEMIQEVSRASGIQIIAATGNHLAVPRPFGDISPSVIAQLYIREIEEGIEGTGIKAGIIKVASRGGITAPQEVVLRAAARAHIHTGLPISTHTWSPDVGEQQVKILEEEGVDLSRVYIGHSNDDTDMDYLLGLLNKGVWLGMDRYGIPGTPNWEQRTEIVKKLIDAGFSHRIMLSHDSVPKARYTLEERRRINPDGYNFIPRKVMPRLKELGASEEVIQQITVENPRRFFE

SEQ ID NO: 32 - protein, 305 aa, unidentified
  source 1..305: note = Bacteria
ASINSVLGPLDTADIGYTLSHEHVMVTSAGIQYVYPEFIDREGSISKGIADLKTAYGEGLRTIVDVTTIDLGRDIRMLEQVSRESGINIICATGVWRDIPRVFWSASPDMVAPLFIREIEEGIEGTGIKAGIIKVANMGGVTAEGEIILRAAARAQKATGVPISTHTWAPEVGEQQVRIFEDEGVDLNRVYVGHSNDTTDTDYLIGLLEKGVWIGLDRYGTEHTPDWIGRTETAKKLIDAGYGHRIMLGHDSVTLSIASKEQRLKYNPDGYLFITRNVVPKLLELGATDEDIQNIFVNNPRNFFE